(19) United States
(12) Patent Application Publication
(10) Pub. No.: US 2026/0130597 A1
(43) Pub. Date: May 14, 2026

(54) SYSTEM AND METHOD FOR CALIBRATIONLESS DETECTION OF BLOOD PRESSURE WITH VIDEO ACQUISITION PLATFORM

(71) Applicant: Aktiia SA, Neuchâtel (CH)

(72) Inventors: Josep Sola, Cormondrèche (CH); Serj Haddad, Belmont sur Lausanne (CH); Calvin Limat, Düdingen (CH); Nikola Tomovic, Belgrade (SR); Dragomir Cvetkovic, Kraljevo (SR); Andreu Arderiu, Lausanne (CH); Sibylle Fallet, Corcelles-pres-Concise (CH); Sasan Yazdani, Vufflens-la-Ville (CH); Bastien Orset, Morges (CH)

(21) Appl. No.: 19/425,853

(22) Filed: Dec. 18, 2025

Related U.S. Application Data
(63) Continuation of application No. PCT/EP2024/081921, filed on Nov. 11, 2024.

(51) Int. Cl. A61B 5/021; A61B 5/00

(52) U.S. Cl. CPC A61B 5/02108; A61B 5/02141; A61B 5/681; A61B 5/6898; A61B 5/7275; A61B 5/742

(57) ABSTRACT

A system for determining a blood pressure value of a user including a sensor adapted to receive a signal from the user and a processor. The processor is programmed to perform operations that include monitoring a safety condition of the sensor, initiating the sensor to collect the signal, monitoring a quality of the signal received from the sensor, generating a compressed signal from the signal, delivering the compressed signal as an input to a model for determining the blood pressure value, and receiving the blood pressure value from the model.

### TECHNICAL FIELD

This specification relates to systems and methods for measuring blood pressure and, in particular, to the determination of blood pressure values using non-invasive sensors.

### BACKGROUND

Blood pressure is typically measured using a device called a sphygmomanometer. Blood pressure measurements are recorded in millimeters of mercury (mmHg) and consists of two values: the systolic pressure and the diastolic pressure. Systolic pressure measures the force in the arteries when the heart beats, while diastolic pressure measures it when the heart is at rest. In most cases, an inflatable cuff is placed around the upper arm, inflated to restrict blood flow, and a stethoscope is used to listen as air is released to determine the systolic and diastolic readings. Due to the need for such specialized equipment, most individuals have their blood pressure measured on a relatively infrequent basis (e.g., when visiting a doctor).

Cuff-free blood pressure measurements can be performed using sensors and algorithms to provide non-invasive readings without using an inflatable cuff. Techniques like photoplethysmography (PPG) and pulse transit time (PTT) may be used. PPG involves the use of optical sensors that track blood volume changes by analyzing how light reflects through the skin, correlating it with heart rate and blood flow. Likewise, PTT measures the time it takes a pulse wave to travel between two body points, such as from the heart to the wrist, and uses this data with heart rate and arterial stiffness to estimate blood pressure. While cuff-free systems may be convenient, their precision often depends on calibration measurements (e.g., captured using an inflatable cuff system). Due to such calibration requirements, it can be difficult for individuals to obtain, and maintain, accurate blood pressure measurements from cuff-free systems.

### SUMMARY

At least one aspect of the present disclosure is directed to a system for determining a blood pressure value of a user. The system includes a sensor adapted to receive a signal from the user and a processor. The processor is programmed to perform operations that include monitoring a safety condition of the sensor, initiating the sensor to collect the signal, monitoring a quality of the signal received from the sensor, generating a compressed signal from the signal, delivering the compressed signal as an input to a model for determining the blood pressure value, and receiving the blood pressure value from the model.

In some embodiments, the sensor comprises a video acquisition system. In some embodiments, the video acquisition system is embedded within a smartphone. In some embodiments, the signal comprises a pulsatility signal. In some embodiments, monitoring a safety condition of the sensor comprises determining a temperature of the sensor. In some embodiments, the processor is programmed to prevent operation of the sensor upon determining that the temperature of the sensor exceeds a predetermined temperature threshold.

In some embodiments, monitoring the quality of the signal received from the sensor comprises (i) evaluating the quality of the signal at a first time period after initiating the sensor and (ii) evaluating the quality of the signal at a second time period after initiating the sensor. In some embodiments, the processor is programmed to prompt the user and restart collection of the signal upon determining that the quality of the signal is below a predetermined quality threshold.

In some embodiments, delivering the compressed signal comprises transmitting the compressed signal to a remote server storing the model. In some embodiments, the processor is programmed to transmit the signal in a format larger than the compressed signal to a remote storage location. In some embodiments, the processor is programmed to present information related to the blood pressure value to the user.

Another aspect of the present disclosure is directed to a system for determining a blood pressure value of a user. The system includes a sensor adapted to receive a signal from the user and a processor. The processor is programmed to perform operations that include educating the user to improve a quality of the signal, initiating the sensor to collect the signal, monitoring the quality of the signal received from the sensor, delivering the signal as an input to a model for determining the blood pressure value, and receiving the blood pressure value from the model.

In some embodiments, educating the user comprises presenting information to the user regarding sensor settings. In some embodiments, educating the user comprises presenting information to the user regarding placement of the sensor. In some embodiments, monitoring the quality of the signal received from the sensor comprises (i) evaluating the quality of the signal at a first time period after initiating the sensor and (ii) evaluating the quality of the signal at a second time period after initiating the sensor. In some embodiments, the first time period occurs during a time period of at least a first full heartbeat of the user. In some embodiments, the second time period occurs during at least a first third of the total signal collection time period. In some embodiments, the processor is programmed to prompt the user and restart collection of the signal upon determining that the quality of the signal is below a predetermined quality threshold. In some embodiments, the processor is programmed to present information related to the blood pressure value to the user.

Another aspect of the present disclosure is directed to a system for determining a blood pressure value of a user. The system includes a sensor adapted to receive a signal from the user and a processor. The processor is programmed to perform operations that include monitoring a safety condition of the sensor, initiating the sensor to collect the signal, delivering the signal as an input to a model for determining the blood pressure value, and receiving the blood pressure value from the model.

In some embodiments, monitoring a safety condition of the sensor comprises determining a temperature of the sensor. In some embodiments, the processor is programmed to prevent operation of the sensor upon determining that the temperature of the sensor exceeds a predetermined temperature threshold. In some embodiments, the processor is further programmed to present information related to the blood pressure value to the user.

Another aspect of the present disclosure is directed to a system for determining a blood pressure value of a user. The system includes a sensor adapted to receive a signal from the user and a processor. The processor is programmed to perform operations that include initiating the sensor to collect the signal, generating a compressed signal from the signal, delivering the compressed signal as an input to a model for determining the blood pressure value, and receiving the blood pressure value from the model.

In some embodiments, delivering the compressed signal comprises transmitting the compressed signal to a remote server storing the model. In some embodiments, the processor is programmed to transmit a second version of the signal having less compression than the compressed signal to a remote storage location. In some embodiments, the second version of the signal is a raw video signal collected by the sensor. In some embodiments, the processor is programmed to present information related to the blood pressure value to the user. In some embodiments, the sensor comprises a camera. In some embodiments, the signal comprises a sequence of images collected by the camera. In some embodiments, generating the compressed signal from the signal includes transforming the sequence of images into at least one pulsatility signal. In some embodiments, the at least one pulsatility signal is a unidimensional pulsatility signal.

Another aspect of the present disclosure is directed to a system for monitoring blood pressure. The system includes a processor and a housing having first sensor adapted to receive a signal from the user, the signal adaptable to be used as an input to a model for determining a blood pressure value of the user. The processor is programmed to perform operations that include executing an application to present information related to the blood pressure value to the user on a user interface and facilitating the user obtaining a second sensor adapted to receive a signal from the user.

In some embodiments, the housing comprises at least one of a smartphone, a tablet, or a smartwatch. In some embodiments, the first sensor comprises a video acquisition system. In some embodiments, the signal comprises a pulsatility signal. In some embodiments, facilitating the user obtaining a second sensor comprises collecting payment from the user for the second sensor. In some embodiments, the second sensor is of a same type as the first sensor. In some embodiments, the second sensor is of a different type than the first sensor. In some embodiments, the first sensor is adapted to collect the signal for determining spot check blood pressure values for the user and the second sensor is adapted to collect the signal determining periodic blood pressure values for the user. In some embodiments, the second sensor comprises a wearable device. In some embodiments, the second sensor comprises a wristband. In some embodiments, the second sensor is an optical sensor housed in one of a wearable device, a smartwatch, or a wristband.

In some embodiments, the processor is programmed to perform operations that include transitioning an account of the user from association with the first sensor to association with the second sensor. In some embodiments, transitioning the account occurs without a need for the user to create a new account or download a separate application. In some embodiments, transitioning the account comprises integrating historical data collected by the first sensor with data collected by the second sensor. In some embodiments, transitioning the account comprises integrating morphological data collected by the first sensor with data collected by the second sensor. In some embodiments, transitioning the account comprises integrating personalized settings associated with the first sensor with personalized settings associated with the second sensor. In some embodiments, transitioning the account comprises integrating a user health profile associated with the first sensor with a user health profile associated with the second sensor. In some embodiments, the first sensor is configured to determine a health condition of the user and the second sensor is configured to manage the health condition of the user. In some embodiments, the health condition of the user is determined based on the blood pressure value of the user.

### BRIEF DESCRIPTION OF THE DRAWINGS

The accompanying figures, which are included as part of the present specification, illustrate the presently preferred embodiments and together with the general description given above and the detailed description of the preferred embodiments given below serve to explain and teach the principles described herein.

FIG. 1 is a block diagram of a blood pressure measurement system in accordance with aspects described herein;

FIG. 2A is a front view of a user device in accordance with aspects described herein;

FIG. 2B is a rear view of a user device in accordance with aspects described herein;

FIG. 3A is a front view of a measurement configuration in accordance with aspects described herein;

FIG. 3B is a rear view of a measurement configuration in accordance with aspects described herein;

FIG. 4 is a view of a measurement configuration in accordance with aspects described herein;

FIG. 5 is a view of a measurement configuration in accordance with aspects described herein;

FIG. 6 is a block diagram of a portion of the blood pressure measurement system of FIG. 1 in accordance with aspects described herein;

FIG. 7 is a flow diagram of a method for monitoring network status during a measurement process in accordance with aspects described herein;

FIG. 8 is a flow diagram of a method for monitoring thermal conditions during a measurement process in accordance with aspects described herein;

FIG. 9 is a flow diagram of a method for determining a blood pressure value of a user in accordance with aspects described herein;

FIG. 10 is a flow diagram of a method for determining a blood pressure value of a user in accordance with aspects described herein;

FIG. 11 is a flow diagram of a method for determining a blood pressure value of a user in accordance with aspects described herein;

FIG. 12 is a flow diagram of a method for determining a blood pressure value of a user in accordance with aspects described herein; and

FIG. 13 illustrates an example computing device.

While the present disclosure is subject to various modifications and alternative forms, specific embodiments thereof have been shown by way of example in the drawings and will herein be described in detail. The present disclosure should not be understood to be limited to the particular forms disclosed, but on the contrary, the intention is to cover all modifications, equivalents, and alternatives falling within the spirit and scope of the present disclosure.

### DETAILED DESCRIPTION

Blood pressure has typically been measured using a sphygmomanometer. In most cases, an inflatable cuff is placed around the upper arm, inflated to restrict blood flow, and a stethoscope is used to listen as air is released to determine the systolic and diastolic readings. Due to the need for specialized equipment, most individuals have their blood pressure measured on a relatively infrequent basis (e.g., when visiting a doctor). However, cuff-free blood pressure measurements may be performed using sensors and algorithms to provide non-invasive readings without using an inflatable cuff. For example, PPG-based techniques involve the use of optical sensors that track blood volume changes by analyzing how light reflects through the skin, correlating it with heart rate and blood flow. Likewise, PTT-based techniques measure the time it takes a pulse wave to travel between two body points, such as from the heart to the wrist, and uses this data with heart rate and arterial stiffness to estimate blood pressure. While these cuff-free systems may be convenient, their precision often depends on calibration measurements (e.g., captured using an inflatable cuff system). Due to such calibration requirements, it can be difficult for individuals to obtain, and maintain, accurate blood pressure measurements from cuff-free systems.

As described above, individuals wishing to use cuff-free systems must first have access to an inflatable cuff system to fulfill the calibration requirements of the cuff-free system. Therefore, worldwide availability of blood pressure readings is dependent on the availability of inflatable cuff systems. There is a clear medical need to deploy, at large scale, blood pressure monitoring systems that are not dependent on individualized access to inflatable cuff systems. By eliminating the initial calibration requirements of cuff-free systems, blood pressure screening and monitoring can be made available to everyone, independent of an individual's location (e.g., rural or non-rural regions) or an individual's access to medical care. Cuff-free systems that provide calibrationless blood pressure measurement are thus a major need in public health. Common devices, such as smartphones with integrated cameras, have the potential to resolve this need.

As such, improved systems and methods for determining blood pressure values using non-invasive sensors are provided herein. In some embodiments, a system is configured to determine accurate blood pressure values without the use of calibration measurements or values. In some embodiments, the system includes a sensor adapted to receive a signal from a user and a processor programmed to perform operations including: monitoring a safety condition of the sensor, initiating the sensor to collect the signal, monitoring a quality of the signal received from the sensor, generating a compressed signal from the signal, delivering the compressed signal as an input to a model for determining the blood pressure value, and receiving the blood pressure value from the model.

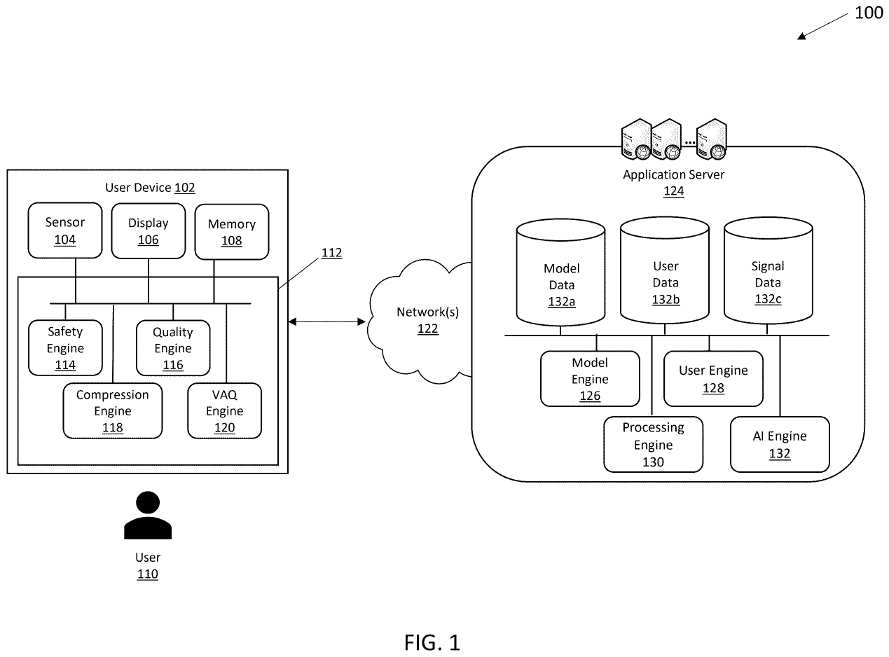

FIG. 1 is a block diagram of a blood pressure measurement system 100 in accordance with aspects described herein. The system 100 includes a user device 102 and an application server 124. In some examples, the user device 102 is a smartphone, a computer, a tablet, a smartwatch, a wearable smart device, a non-wearable smart device, a smart device embedded within another item or device, or a dedicated medical device. Other types of user devices are possible. In some examples, user device 102 includes at least one sensor 104, at least one display 106, and at least one memory element 108. The user device 102 may have a housing that includes one or more sensors. The sensor 104 is configured to receive (or collect) a signal from a user 110. In some examples, the signal is a pulsatility signal. The pulsatility signal may be a PPG signal from a photoplethysmographic sensor, or any other pulsatility sensor or array of sensors, such as, but not limited to a camera sensor, a photo-diode sensor, a bioimpedance sensor, an ultra-sound sensor, a magnetic sensor, a radar sensor, a sensor based on radio frequency, a mechanical sensor, a volume sensor, a non-invasive sensor, an invasive sensor, an intra-arterial sensor, a minimal invasive sensor, a subcutaneous sensor, a tonometer sensor, a strain sensor, a plethysmographic sensor, a microphone, a capacitive sensor, an electromagnetic sensor, a Raman sensor, or any sensor capable of measuring a pulsatility signal either from a capillary bed of the skin or from any other section of the arterial tree. In some examples, the pulsatility signal is a unidimensional pulsatility signal. In some examples, the signal is derived from an image, a series of images, or a video captured by the sensor 104. The sensor 104 may alternatively be referred to as the camera 104.

In some examples, a client application 112 is configured to run (or operate) on the user device 102. The client application 112 may be configured to run in a web browser or a special-purpose software application executing on the user device 102 (e.g., a smartphone application). In some examples, the client application 112 includes a safety engine 114, a quality engine 116, a compression engine 118, and a video acquisition engine (VAQ) 120. The client application 112 is configured to process, at least in part, the signal received by the sensor 104 to determine blood pressure values of the user 110. The user device 102 (or the client application 112) is configured to communicate with the application server 124 through one or more data communication networks 122 such as the Internet, for example. The client application 112 may communicate with the application server 102 over the network 122 using Hypertext Transfer Protocol (HTTP), another standard protocol, or a proprietary protocol, for example. The application server 124 provides functionality for processing data (e.g., pulsatility signals) and storing data (e.g., user data, signal data, etc.).

The application server 124 comprises software components and databases that can be deployed at one or more data centers (not shown) in one or more geographic locations, for example. The application server 124 software components include a model engine 126, a user engine 128, a processing engine 130, and an artificial intelligence engine 132. The software components can comprise subcomponents that can execute on the same or on a different individual data processing apparatus. The application server 124 databases include a model data database 132a, a user data database 132b, and a signal data database 132c. The databases can reside in one or more physical storage systems. Example features of the software components and data processing apparatus will be further described below. It should be appreciated that the application server 124 may include or otherwise support additional engines and tools. For example, the application server 102 may support third-party tools that provide additional functions to the engines 126-132 and/or the databases 132a-c. In some examples, the application server 124 is configured to utilize one or more Application Programming Interfaces (APIs) to communicate with third-party tools and engines.

Although this application will describe many functions as being performed by client application 112, in various implementations, some or all functions performed by the client application 112 may be performed remotely by the application server 124. Likewise, in various implementations, some or all functions performed by the application server 124 may be performed locally by the client application 112.

### Measurement Process

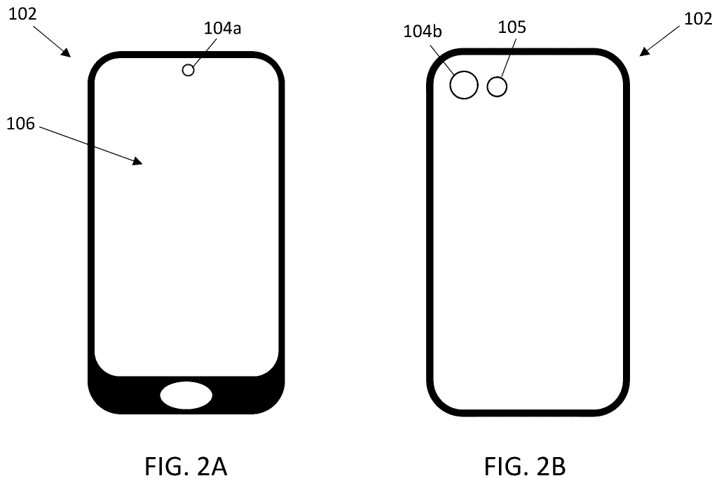

FIGS. 2A, 2B illustrate an example user device 102 that is used to capture measurements in accordance with aspects described herein. FIG. 2A illustrates a front view of the user device 102 showing the display 106 and a front camera 104a (i.e., a front-facing camera). FIG. 2B illustrates a back view of the user device 106 showing a rear camera 104a (i.e., a rear-facing camera) and a flash module 105. In some examples, the measurement process is performed by having a part of the user's body cover the lens of the camera 104a or 104b (e.g., by instance putting the fingertip on top of the camera). As will be described below, the measurement process may be performed in both display-side up and display-side down configurations.

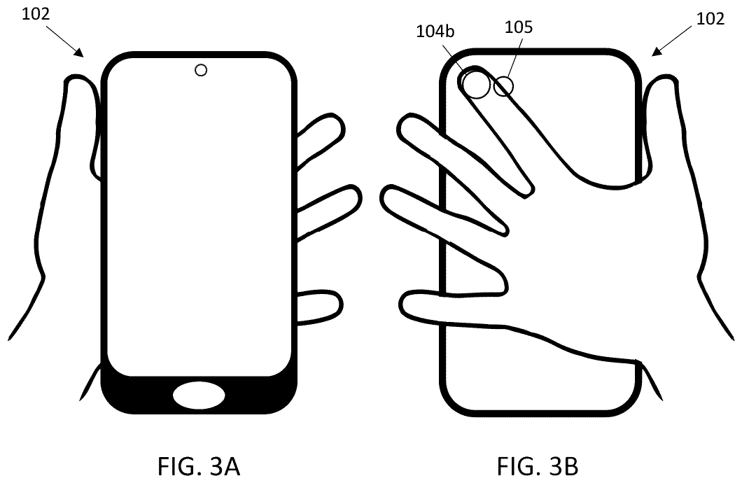

FIGS. 3A, 3B illustrate a first measurement configuration in accordance with aspects herein. As shown, the user device 102 is held in the hand of the user 110 display-side up. The user's finger (e.g., index finger) is positioned on top of the rear camera 104b. In some examples, the user's finger is positioned such that it does not cover the flash module 105. In some examples, the user's finger may partially cover or fully cover the flash module 105. In some examples, the flash module 105 is turned on during the measurement process (i.e., while the camera 104b is collecting images/video). In some examples, the flash module 105 is turned off during the measurement process. In some examples, the flash module 105 is turned on and off during the measurement process.

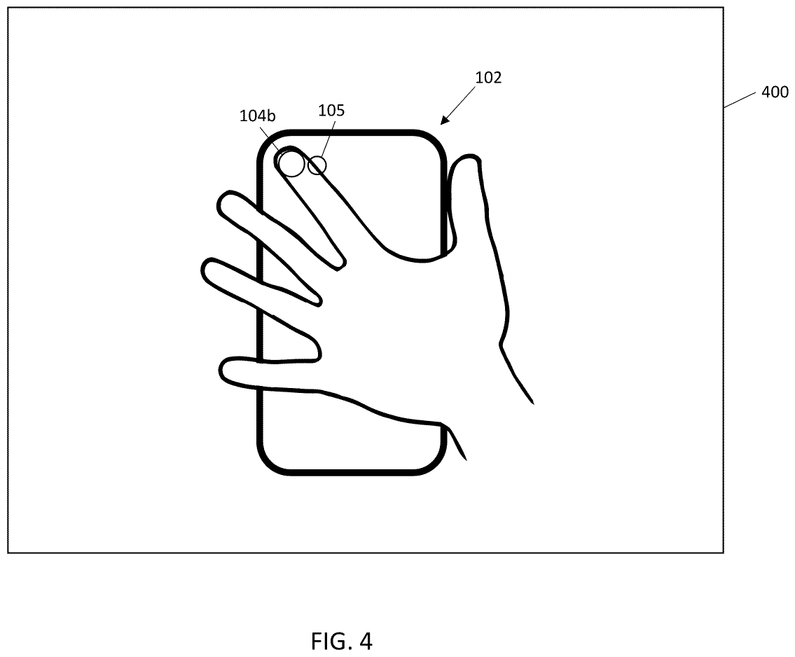

FIG. 4 illustrates a second measurement configuration in accordance with aspects herein. As shown, the user device 102 is placed display-side down on a flat surface 400. The surface 400 may be a table, countertop, or any other flat surface. The user's finger is positioned on top of the rear camera 104b. In some examples, the user's finger is positioned such that it does not cover the flash module 105. In some examples, the user's finger may partially cover or fully cover the flash module 105. In some examples, the flash module 105 is turned on during the measurement process (i.e., while the camera 104b is collecting images/video). In some examples, the flash module 105 is turned off during the measurement process. In some examples, the flash module 105 is turned on and off during the measurement process. This orientation provides a stable and secure setup for the measurement process, minimizing the potential for device movement that could interfere with signal acquisition. By having the display face down, the user 110 can comfortably position their fingertip over the lens of the rear camera 104b and the flash module 105 without needing to hold the device 102, thereby reducing hand tremors or unintentional movements. This hands-free approach enhances the consistency and reliability of the measurements by ensuring that the device 102 remains stationary throughout the procedure.

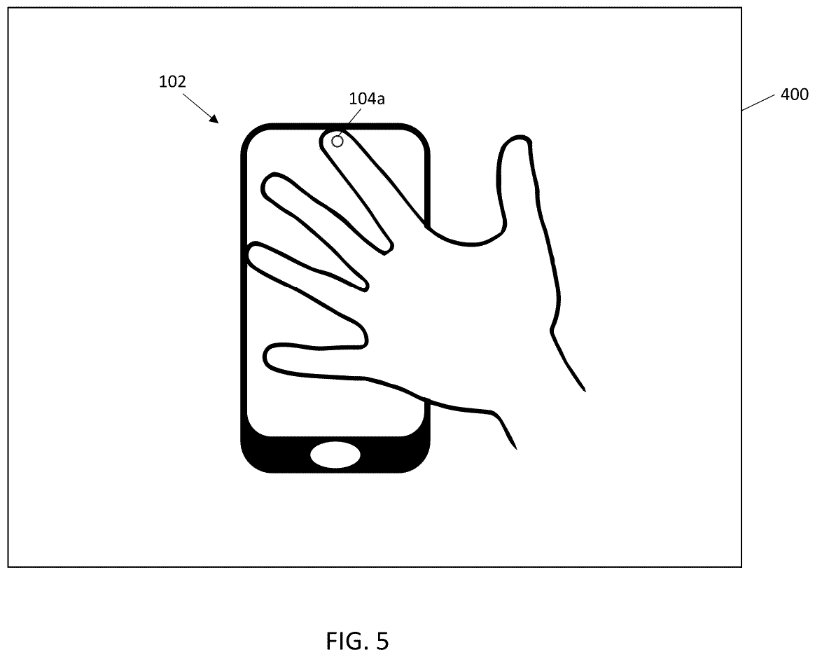

FIG. 5 illustrates a third measurement configuration in accordance with aspects herein. As shown, the user device 102 is placed display-side up on the flat surface 400. The user's finger is positioned on top of the front camera 104a. In some examples, the user's finger (or hand) is positioned such that it does not cover the display 106. In some examples, the user's finger (or hand) may partially cover or fully cover the display 106. In some examples, the display 106 is illuminated (or brightened) during the measurement process (i.e., while the camera 104a is collecting images/video). In some examples, the display 106 is turned off (or darkened) during the measurement process. In some examples, parameters (or content) of the display 106 are varied during the measurement process. For example, the brightness of the display 106 or the colors displayed may be varied during the measurement process. In some examples, specific regions of the display 106 are varied (e.g., in brightness or color). In some examples, the parameters, characteristics, and/or content of the display 106 are varied such that the user's finger is sufficiently illuminated in order to achieve a desired quality of the signals received from the user 110. In some examples, the quality engine 116 is configured to adapt the parameters, characteristics, and/or content of the display 106 in a real-time feedback loop. In some examples, the display 106 is used to present visual cues (e.g., text pop-ups, arrows and indicators) to guide the user 100 in positioning their finger in the right location. In some examples, the display 106 is used to present game-like content to obtain optimum pressure, color changing feedback (e.g., for pressure), and/or real-time camera feedback such that the user 100 can see which camera to put their finger on, if it's well placed, etc.

To assist users in achieving optimal finger placement over the lens of the camera 104a or 104b, the system 100 utilizes haptic feedback mechanisms. When the user's fingertip fully covers the camera lens and proper contact is established, the device 102 provides a subtle vibration signal. This haptic feedback confirms to the user 110 that their finger is correctly positioned, eliminating the need for visual confirmation. The tactile cue is immediate and intuitive, allowing users to adjust their finger placement based on physical sensations alone. This feature is particularly beneficial for users who may have visual impairments or in situations where looking at the display 106 is not convenient.

The measurement process is further guided by audio cues that offer step-by-step instructions to the user 110. These auditory prompts provide real-time guidance on maintaining the correct pressure and finger position, ensuring that the user 110 applies neither too much, nor too little force, on the lens of the camera 104a or 104b. By delivering clear and concise instructions through sound, the system 100 enables users to focus on achieving optimal measurement conditions without needing to observe the display 106. This hands-free guidance is especially useful in the display-side down setup, as it allows users to rely entirely on auditory feedback to navigate the measurement process.

In addition to instructional cues, the system 100 employs audio signals to indicate key stages of the measurement, such as the start, progress milestones, and completion of the procedure. For example, a distinct tone may signify the initiation of data recording, while another sound might indicate that the measurement is halfway complete. Upon completion, a final audio cue confirms that the process has concluded successfully. These auditory markers keep the user 110 informed of the measurement's status, enhancing their awareness and engagement without requiring visual attention. This continuous auditory communication ensures that the user 110 remains synchronized with the measurement process from start to finish.

Upon the completion of the measurement, the client application 112 automatically turns off the camera flash, providing a visual indication that the process has ended without necessitating any manual intervention from the user 110. The deactivation of the flash serves as a clear and immediate signal that the user 110 can remove their finger from the lens of the camera 104. This automated response enhances the convenience of the measurement procedure by eliminating the need for the user 110 to interact with the device to conclude the session. It also conserves battery life by ensuring that the flash is not left on longer than necessary.

By combining haptic, audio, and visual feedback mechanisms, the system 100 delivers an effortless and hands-free user experience throughout the measurement procedure. Haptic feedback ensures correct finger placement, audio cues provide continuous guidance and progress updates, and visual signals like the automatic flash deactivation confirm the measurement's completion. This multimodal approach caters to different sensory preferences and enhances accessibility, making the measurement process intuitive and user-friendly. It maintains precision and consistency by guiding the user 110 at every step without requiring constant visual monitoring or manual input, thereby improving the accuracy and reliability of the blood pressure readings obtained. By implementing these variants in the measurement steps, the system 100 accommodates diverse user needs and preferences, ensuring that the blood pressure monitoring process is accessible, efficient, and effective across various contexts and environments.

In some examples, measurement process incudes using the camera 104 to acquire a series of the images (or video) without contacting the user's body. For example, the camera 104 may be used to film a video of the user's entire face, a part of the face, the entire body, and/or part of the body. In such examples, the camera 104 records a series of images of the user 110 (e.g., the entire face, part of the face, the entire body, or a part of the body) from which the signal is extracted.

To determine the user's blood pressure, the user device 102 transmits at least a portion of the data collected by the sensor 104 to the application server 124 for processing. In some examples, the user device 102 is configured to transmit a single signal extracted from the frames captured by the sensor 104. For example, the client application 112 may extract a signal representing the entire frame to be transmitted to the application server 124. In some examples, the user device 102 transmits two or more signals extracted from the frames captured by the sensor 104. For example, the client application 112 may extract signals representing different regions of interest (ROIs) of each frame to be transmitted to the application server 124. In some examples, the user device 102 transmits a compressed version of video captured by the sensor 104 to the application server 124. In some examples, the user device 102 transmits raw video captured by the senor 104 to the application server 124.

### Client Application

A video acquisition system of the user device 102 is configured to capture pulsatility signals from the user 110. In some examples, the video acquisition system 100 includes the sensor 104 of the user device 102 and the VAQ engine 120 of the client application 112. The VAQ engine 120 is configured to control and operate the sensor 104 to capture the signals from the user 110. The VAQ engine 120 may use native software functions of an operating system of the device 102 to control and operate the sensor 104. In some examples, the video acquisition system includes the display 106 of the user device 102. The display 106 may be used to view (or preview) the images or video being recorded to capture the signals. The video acquisition system is used to capture signals from a particular region of the user's body (e.g., a fingertip). In some examples, the sensor 104 is held in proximity to the region or pressed against the region to capture signals from the user 110. In some examples, the sensor 104 is configured to capture signals from the user 110 without contacting the user's body (e.g., by capturing an image/video of the user's face, body, etc.).

The video acquisition system of the user device 102 is configured to function effectively with or without artificial lighting capabilities. In environments where ambient lighting is sufficient, the sensor 104 can capture high-quality pulsatile signals from the user 110 (e.g., via the user's fingertip) without additional illumination. This adaptability ensures that users can obtain accurate blood pressure measurements in a variety of settings, whether indoors under artificial light or outdoors in natural daylight, enhancing the convenience and flexibility of the system 100. In scenarios where ambient lighting is inadequate, the system 100 may leverage a built-in flash light of the user device 102 as an artificial lighting source (e.g., the camera flash source). By illuminating the user's fingertip during the recording process, the flash light enhances the discernibility of blood flow characteristics captured by the sensor 104. In some examples, the client application 112 is configured to control the flash intensity, focal point of the flash, and timing to optimize signal quality while ensuring user comfort and safety, thereby enabling precise blood pressure measurements even in low-light conditions.

In some examples, the sensor 104 includes a front-facing camera (e.g., camera 104a). When utilizing front camera modules for signal acquisition, the client application 112 may employ the display 106 of the user device 102 as an artificial lighting source by adjusting its brightness, and focal point—either partially or completely. By increasing screen luminance, the user's fingertip may be uniformly illuminated when placed over the front camera modules, improving the detection of pulsatile signals. This method eliminates the need for external light sources and allows for a user-friendly experience, as the screen's brightness can be modulated to balance signal quality with power consumption and user comfort.

The video acquisition system may be compatible with other artificial lighting sources beyond the native capabilities of the user device 102. Such sources may include external devices, such as clip-on LED lights, ring lights, or specialized cases with integrated illumination designed to enhance sensor performance. By supporting a variety of lighting accessories, the system 100 ensures that users can achieve optimal signal quality under diverse conditions and with different device models, thereby expanding the usability and accessibility of the blood pressure measurement feature.

Various user devices, such as smartphones, may exhibit diverse configurations of back camera and flash distributions, which the system 100 accommodates to optimize performance. For example, certain smartphone models feature a single, dual, or triple-camera setup with the flash positioned between or adjacent to the lenses. Likewise, some smartphone models include multiple flash modules. The VAQ engine 120 provides tailored guidance for each device configuration. For example, the VAQ engine 120 may provide visual content and instructions (e.g., via the display 106) to ensure that users correctly position their fingertip over the camera and flash components. In some examples, the VAQ engine 120 provides visual feedback to the user 110 to correct the positioning of their fingertip with respect to the camera(s) 104. In some examples, the VAQ engine 120 is configured to provide audio content to the user 110 such that the user 110 can receive instructions and feedback without needing to view the display 106. By accounting for these variations, the VAQ engine 120 maintains consistent signal quality across different devices.

Front camera configurations also vary among smartphone models, influencing how screen illumination is utilized. For example, certain smartphone models integrate the front camera alongside sensors within the display (or screen) area, allowing for targeted brightness adjustments in that region. Other devices feature punch-hole or under-display cameras, requiring different screen illumination strategies. The VAQ engine 120 is configured to adapt to these variations by modulating screen brightness appropriately, ensuring effective fingertip illumination and high-quality signal capture regardless of the front camera arrangement.

Some smartphones have a camera module that protrudes from the device's back surface, creating a physical protuberance. This design element can affect how the user's fingertip interfaces with the camera and flash during measurement. The VAQ engine 120 addresses this by providing specific instructions and visual aids to help users properly position their fingertip over protruding camera modules. For example, the VAQ engine 120 may recommend adjusting the finger angle or applying gentle pressure to ensure full contact, thus mitigating potential challenges posed by the device's physical characteristics.

Camera lens configurations, such as lens diameter and the presence of multiple lenses, can influence the area of contact and the quality of the captured signals. Larger lenses may allow for greater contact area and improved signal acquisition, while smaller lenses require more precise fingertip placement. The VAQ engine 120 incorporates adaptive algorithms that adjust to the lens specifications of each smartphone model. It provides customized user guidance to accommodate single, dual, or multi-lens setups, ensuring that the pulsatile signals are consistently and accurately captured across different camera configurations.

By integrating examples of various user device designs and their respective features, the VAQ engine 120 provides a high level of compatibility and robustness. Whether dealing with an devices equipped with a triple-lens rear camera and a true-tone flash, or a device featuring a high-resolution front camera with edge-to-edge display illumination, the VAQ engine 120 can adapt its operation accordingly. As such, the VAQ engine 120 (and the system 100) can provide precise blood pressure measurements across a wide spectrum of devices, accommodating differences in hardware layouts, sensor technologies, and user interaction modalities.

While some of the above examples describe the user device 102 as a smartphone, it should be appreciated that the user device 102 may be a different type of device. For example, the user device 102 may be any device equipped with a digital camera and sufficient processing capabilities, such as tablets, smartwatches, laptops, or dedicated medical devices. In some examples, the user device 102 is a display-less device. This versatility allows for a wide range of applications, extending the benefits of non-invasive blood pressure monitoring to various platforms. By leveraging the ubiquitous presence of digital cameras in modern devices, the system 100 ensures that users have accessible and convenient options for health monitoring.

In some examples, the VAQ engine 120 includes a control mechanism that verifies whether the user device 120 in use is supported by referencing a predefined list of compatible devices. In some examples, the list is compiled based on thorough testing and validation to ensure that each listed device meets the necessary hardware and software requirements for accurate blood pressure measurement. When the user 110 launches the client application 112, the VAQ engine 120 accesses the list to confirm device compatibility. If the device model matches an entry on the list, the user 110 is allowed to proceed with the measurement process. Otherwise, the VAQ engine 120 notifies the user that their device is unsupported, preventing potential inaccuracies and ensuring the integrity of the measurement results. This methodical approach guarantees that only devices capable of delivering reliable performance are utilized within the system 100.

To streamline the user experience, the VAQ engine 120 may automatically detect the smartphone model without requiring any manual input from the user 110. In some examples, upon initialization, the VAQ engine 120 retrieves device-specific information using built-in APIs provided by the device's operating system. This information may include the device's model identifier, hardware specifications, sensor (or camera) capabilities, and current operating system version. By automatically gathering these details, the VAQ engine 120 can promptly assess whether the device 102 is supported. This automated verification process minimizes user effort, reduces the likelihood of errors associated with manual data entry, and ensures that only compatible devices are used for blood pressure measurement, thereby maintaining high standards of accuracy and reliability.

In some examples, the client application 112 (or the VAQ engine 120) is configured to dynamically manage the list of supported devices, allowing the list to adapt to new devices and software updates in real-time. In some examples, the client application 112 connects with a remote server (e.g., the application server 124) to retrieve the most recent compatibility data whenever the client application 112 is launched. In some examples, the most recent compatibility data is retrieved by the VAQ engine 120 at scheduled intervals. As new device models are released, or existing devices receive software updates that affect their performance with the system 100, the compatibility list is updated accordingly on the application server 124. This ensures that users with newly supported devices can access the features of the system 100 without delay, and any devices that become incompatible due to changes in specifications are promptly identified. By dynamically updating the compatibility information, the system 100 maintains its effectiveness and reliability over time, providing users with continuous support and up-to-date functionality.

In some examples, the client application 112 includes pre-programmed educational modules designed to instruct users on the optimal procedures for obtaining accurate blood pressure measurements. These modules provide comprehensive guidance on factors such as proper finger placement over the camera lens, appropriate pressure application, ideal body posture, and environmental conditions conducive to high-quality signal acquisition. By delivering standardized instructions, the client application 112 ensures that all users receive consistent and essential information necessary for effective use. The pre-programmed content can be accessed at any time, allowing users to familiarize themselves with the process before initiating a measurement, thereby enhancing user confidence and proficiency.

In some examples, the client application 112 offers an interactive educational feature that allows users to engage in a simulation mode for practicing finger placement and pressure application. This mode utilizes real-time signal acquisition to provide immediate feedback on the quality of the optical readings as the user adjusts their finger positioning. By visualizing signal strength and quality indicators dynamically (e.g., via the display 106), users can intuitively learn how their actions affect the measurement process. This hands-on practice environment enables users to refine their technique before performing an actual blood pressure reading, significantly reducing the likelihood of errors and improving the reliability of subsequent measurements.

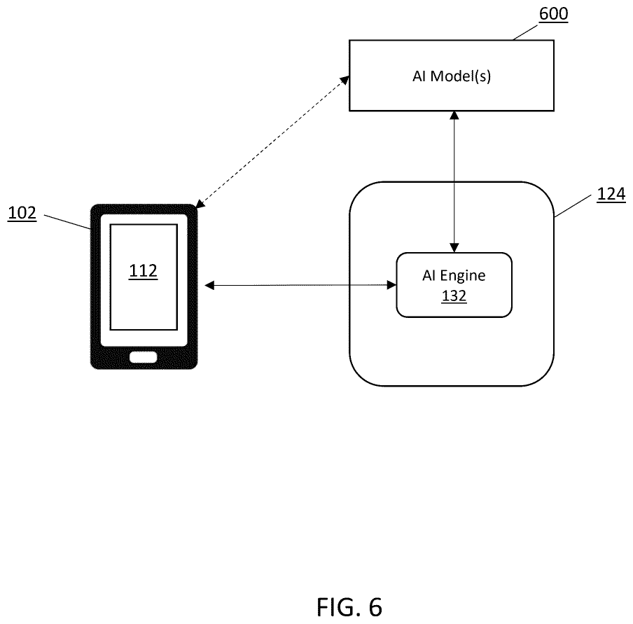

In some examples, the client application 112 is configured to leverage advanced language learning models (LLMs) and AI to generate additional user content. As shown in FIG. 6, the AI engine 132 of the application server 124 may interface with at least one AI model 600. In some examples, the AI model 600 is an LLM. In some examples, the AI model is an internal model that runs on the application server 124. In some examples, the AI model 600 is an external model that the AI engine 132 communicates with via one or more APIs. In some examples, the AI model 600 is a foundational model. In some examples, the AI model 600 is a specialized model that is trained solely for use with the system 100.

In some examples, the client application 112 is configured to interact with the AI model 600 via the AI engine 132. For example, the client application 112 may transmit prompts to the AI engine 132 that are forwarded to the AI model 600. In such examples, the AI engine 132 may forward the answers/content from the AI model 600 to the client application 112. In some examples, the client application 112 transmits signals, information, or parameters to the AI engine 132 and the AI engine 132 generates prompts corresponding to the received signals, information, or parameters. In some examples, the client application 112 can interact with the AI model 600 directly (e.g., via one or more APIs).

The client application 112 may utilize the AI model 600 to generate personalized tutorials that adapt to each user's specific configuration and behavior. By analyzing live data acquisition and user interactions, the AI model 600 can create dynamic educational content that addresses individual learning needs and potential challenges. This personalized guidance may include tailored instructions, tips for improvement, and adaptive feedback that evolves with the user's proficiency. The AI-driven approach ensures that education is not only informative but also highly relevant to the user's unique circumstances, thereby enhancing the overall effectiveness of the training process.

By integrating comprehensive educational resources—including pre-programmed modules, interactive simulation modes, and AI-generated personalized tutorials—the system 100 significantly increases the likelihood that users will perform efficient recordings of high quality. Educated users are better equipped to understand the importance of proper technique and are more likely to adhere to best practices during measurements. This thorough preparation minimizes common errors associated with user handling and environmental factors, leading to more reliable and accurate blood pressure readings. Ultimately, the emphasis on education serves to enhance the system's overall performance and user satisfaction.

Effective education not only improves measurement accuracy but also enhances the perceived value of the data delivered to the user 110. When users understand the process and contribute to the quality of their readings, they are more likely to trust and value the blood pressure information provided by the system 100. This trust is crucial for encouraging regular use and adherence to health monitoring routines. By empowering users through education, the system 100 fosters a sense of engagement and ownership over personal health data, thereby maximizing the utility and impact of the information delivered.

In some examples, the system 100 includes functionality to collect morphological (morpho) data directly from the user 110 through a structured form within the client application 112. This form prompts the user 110 to input personal physiological characteristics that are useful in calibrating the predictive models used in blood pressure estimation. By providing a user-friendly interface for data entry, the client application 112 ensures that users can easily supply accurate information, which enhances the personalization and accuracy of the blood pressure measurements. In some examples, the morphological data collected by the client application 112 encompasses a wide range of physiological attributes relevant to cardiovascular health. Such attributes include, but is not limited to, the user's weight, height, age, and sex. Additional data points may include body mass index (BMI), ethnicity, body fat percentage, blood type, and other biometric indicators. Collecting comprehensive morpho data allows the predictive models of the system 100 to account for individual differences, leading to more accurate and personalized blood pressure readings.

In some examples, to enhance convenience and ensure data accuracy, the system 100 is capable of importing morphological data from third-party sources with the user's consent. This integration may involve syncing with electronic health records, fitness trackers, health apps, or wearable devices that the user 110 already utilizes. By accessing verified data from external sources, the system 100 reduces the need for manual data entry, minimizes user effort, and maintains up-to-date information for more precise blood pressure estimation. In some examples, the user engine 128 of the application server 124 is configured to collect the external morphological data of the user 110 and store it in a database (e.g., the user data database 132b). In such examples, the client application 112 may receive the external morphological data of the user 110 from the application server 124 as it becomes available. In some examples, the client application 112 may query the application server 124 periodically for data updates (e. g, daily, each time the user 110 opens the client application 112, etc.).

In some examples, the client application 112 provides mechanisms for users to input additional health-related information that could influence blood pressure readings and enhance model training. Users can enter details about medications they are taking (e.g., antihypertensives, diuretics, or other drugs affecting cardiovascular function), existing health conditions (e.g., hypertension, diabetes, heart disease), and lifestyle factors (e.g., smoking status, alcohol consumption, and exercise habits). By incorporating this information, the predictive models of the system 100 can adjust for these variables, leading to more accurate and individualized blood pressure assessments.

Beyond user-inputted data, the client application 112 is equipped to automatically recognize and collect additional information pertinent to model training. This includes detecting environmental factors like ambient light conditions during measurement, which can affect signal quality. The client application 112 may also analyze skin type characteristics (e.g., pigmentation levels or skin texture) through image processing techniques. By automatically gathering and incorporating these variables, the client application 112 enhances the robustness of the predictive models of the system 100, allowing them to compensate for factors that might otherwise introduce errors into the blood pressure estimation.

In some examples, the client application 112 includes a feature that allows guest users to perform blood pressure measurements on the user device 102 without altering the primary user's stored data (e.g., stored in memory element 108 or user data database 132b). In some examples, the “guest mode” is achieved by permitting temporary changes to the morphological and personal information for a single measurement session. After the session, the client application 112 reverts to the original user's profile settings. This functionality enables multiple individuals to use the system 100 for occasional measurements, making it versatile for family use or in situations where sharing the device 102 is necessary, while preserving data integrity for the user 110.

In some examples, the client application 112 transforms the process of collecting morphological data into a comprehensive “Daily Health Check-In” routine. This approach encourages users to engage in a holistic health assessment by combining physical biometrics with reflections on mental and emotional well-being. The client application 112 may prompt users to consider factors such as stress levels, mood, sleep quality, and overall mental state alongside their physiological measurements. By integrating mind and body metrics, the system 100 not only enhances the richness of the data collected for predictive model training, but also promotes user mindfulness about their overall health, potentially leading to better health outcomes through increased self-awareness and proactive health management.

### Application Server

The application server 124 is configured to manage user accounts securely and efficiently, forming the backbone of the system's cloud-based services. In some examples, the application server 124 employs a multi-tiered infrastructure that includes application-specific servers, database-specific servers, and authentication services. In some examples, load balancers distribute incoming traffic evenly across the servers to ensure high availability and scalability. The server architecture leverages secure APIs for communication between the client application 112 and the application server 124, utilizing encryption protocols such as TLS to protect data in transit. Firewalls and intrusion detection systems safeguard against unauthorized access, while user data is stored in encrypted databases (e.g., user data database 132b) with secure access controls. This robust architecture ensures seamless user account creation, authentication, and management, providing users with a reliable and secure experience.

Upon receiving the compressed pulsatility signals from the user device 102, the processing engine 130 performs advanced signal processing to prepare the data for blood pressure estimation via the model engine 126. In some examples, this involves aggregating multiple signals if available and conducting pre-processing steps such as de-noising, baseline correction, and normalization (e.g., via the processing engine 130). Techniques such as digital filtering may be applied to remove artifacts and enhance signal quality. The signals are then segmented into appropriate time windows, and feature extraction methods are used to identify key physiological markers relevant to blood pressure, such as pulse transit time, heart rate variability, and waveform morphology. This meticulous pre-processing ensures that the input to the predictive models is of high fidelity, enabling accurate and consistent blood pressure calculations by the model engine 126.

The blood pressure estimation by the model engine 126 relies on a sophisticated model architecture developed using ML techniques tailored for physiological signal analysis. In some examples, the model architecture consists of deep neural networks with layers optimized for temporal and spatial pattern recognition, such as convolutional and recurrent neural networks. The models are trained on large datasets to capture complex relationships between the pulsatility signals and corresponding blood pressure values. In some examples, the training datasets are stored in the model data database 132a. Regularization methods and cross-validation are employed to prevent overfitting and to enhance generalizability. The architecture is designed to be modular, allowing for updates and improvements as new data becomes available, and to accommodate different input types and configurations.

In some examples, the model engine 126 maintains a comprehensive database that stores annotated signals (e.g., the signal data database 132c), where each pulsatility signal is linked with corresponding blood pressure measurements and metadata such as time stamps, user demographics, and environmental conditions. These annotations enable supervised learning, allowing the predictive models to learn accurate mappings between input signals and blood pressure value outputs. The annotated dataset serves as a valuable resource for refining the predictive algorithms, identifying patterns, and improving model robustness. It supports ongoing research and development efforts aimed at enhancing the system's capability to deliver precise and personalized blood pressure estimates.

In addition to annotated data, the signal data database 132c may store non-annotated signals generated during routine use of the system 100. These signals, while not immediately linked to known blood pressure values, provide a rich source of information for unsupervised learning and exploratory data analysis. They help in identifying new patterns, understanding user behavior, and detecting trends that may not be apparent from annotated data alone. Incorporating non-annotated signals allows the system 100 to adapt to a wider range of physiological variations and environmental factors, ultimately contributing to the continuous improvement of the predictive models. The accumulation of both annotated and non-annotated signals enables the models to evolve and adapt, enhancing their accuracy and reliability over time. This iterative improvement process ensures that users receive increasingly precise blood pressure measurements, justifying any initial inconveniences and reinforcing the system's value proposition.

### Network Check

In some examples, the blood pressure predictive model of the system 100 is configured to operate locally on the user device 102. In some examples, the predictive model of the system 100 is configured to operate on the application server 124 (e.g., within the model engine 126). When the predictive model is not executed locally on the user device 102, the quality engine 116 of the client application 112 may perform a network performance check to verify that a sufficient connection exists between the user device 102 and the application server 124. By operating the predictive model on the application server 124, the system 100 safeguards proprietary algorithms from unauthorized access. Additionally, the predictive model may be computationally intensive, requiring processing power beyond the capabilities of the user device 102. By offloading the processing to the application server 124, which is equipped with the necessary computational resources, it can be ensured that the BP predictive model operates efficiently and accurately. Checking performance of the network 122 to determine the current network conditions allows the client application 112 to verify that the user's data exchange can be supported without significant delays or interruptions. Incorporating this check helps maintain the system's low latency objectives and ensures a seamless user experience by confirming that remote processing is feasible before initiating the measurement procedure.

In some examples, consistent network connectivity enables the frames, video, or signals captured during the measurement process to be sent to the application server 124 for algorithmic processing (e.g., by the model engine 126). In some examples, the processing requirements of the predictive model(s) exceed the processing capabilities of the user device 102. By utilizing the application server 124 for data processing, the system 100 leverages enhanced computational power to perform complex analyses necessary for accurate blood pressure determination. To ensure that this server-client interaction functions correctly, the quality engine 116 may continuously monitor the status of the network 122 during the measurement session. If the network 122 is detected to be unavailable or insufficient (e.g., due to low bandwidth or high latency), the quality engine 116 proactively alerts the user 110 by displaying an error screen on the display 106. This immediate feedback prevents the user 110 from proceeding with a measurement that cannot yield valid results due to connectivity issues. By implementing real-time network monitoring, the system 100 enhances reliability and user satisfaction, ensuring that measurements are only conducted when the necessary network conditions are met for successful data transmission and processing.

In some examples, the quality engine 116 is configured to use one or more native functions of the user device's operating system to perform the network check. For example, on Apple iOS devices, the quality engine 116 may be configured to use the Network framework (or Reachability framework) to monitor network connectivity and performance. The quality engine 116 checks if the network status is satisfied and may perform small data transfers to measure bandwidth and latency, ensuring conditions are suitable for data transmission. Likewise, on Google Android devices, the quality engine 116 may be configured to employ the ConnectivityManager and NetworkCapabilities classes to assess network status and performance. The quality engine 116 verifies internet connectivity and evaluates network properties, like bandwidth and latency, to determine if the network 122 can support the measurement process. In cross-platform frameworks, like React Native or Flutter, the quality engine 116 may be configured to use modules such as “NetInfo” or “connectivity_plus” to check network connectivity and listen for changes, ensuring consistent performance checks across various device platforms.

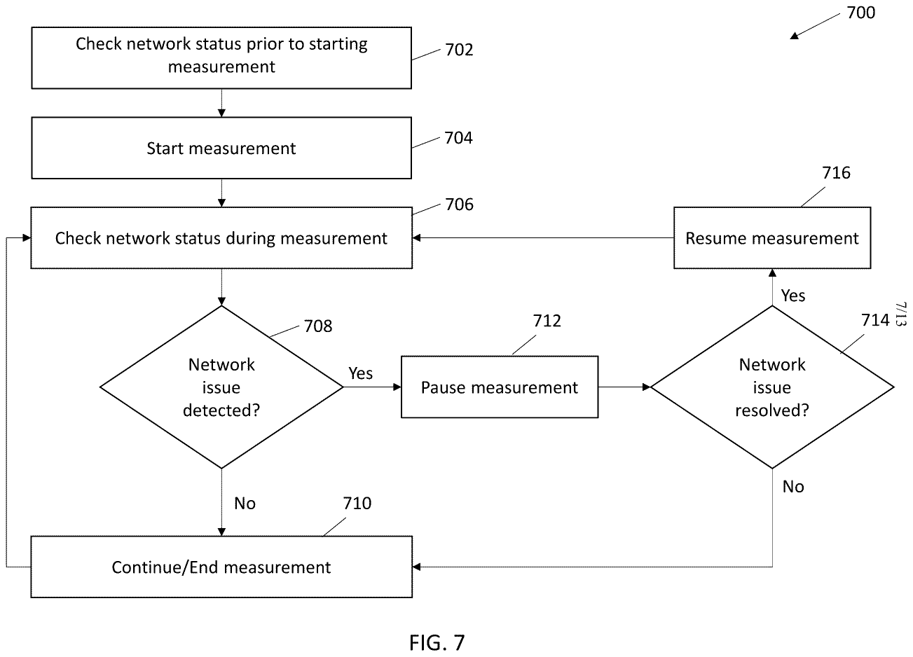

FIG. 7 illustrates a flow diagram of a method 700 for monitoring network status during the measurement process in accordance with aspects described herein. In some examples, the quality engine 116 of the client application 112 is configured to perform the method 700, at least in part. The measurement process corresponds the collection of signals (or the images/videos from which the signals are derived) via the user device 102.

At step 702, the quality engine 116 checks the initial network status of the network 122 prior to starting the measurement process. In some examples, the quality engine 116 determines whether the network 122 meets minimum thresholds for bandwidth and/or latency. If the network 122 does not meet the minimum threshold(s), the quality engine 116 may alert the user (e.g., via the display 106). In some examples, the measurement process is automatically terminated if the network 122 fails to meet the minimum threshold(s).

At step 704, in response to a determination that the network 122 does meet the minimum threshold(s), the quality engine 116 starts the measurement process. In some examples, the quality engine 116 initiates the measurement process by sending one or more messages to the VAQ engine 120. The VAQ engine 120 begins to collect frames from the user 110 that are transmitted, via the network 122, to the application server 124 for processing. In some examples, the data transmitted between the user device 102 and the application server 124 is encrypted using one or more secure protocols (e.g., HTTPS). In some examples, server certificates are validated to protect the sensitive nature of the data being collected and transmitted.

At step 706, the quality engine 116 checks the network status of the network 122 during the measurement. In some examples, the quality engine 116 is configured to check the network status at periodic interval (e.g., every second, every 5 seconds, every 30 seconds, etc.). In some examples, the quality engine 116 is configured to check the network status at specific frame intervals (e.g., every other frame, every 5 frames, every 30 frames, etc.). In some examples, the quality engine 116 is configured to check the network status at certain points in the measurement (e.g., at the 25% complete mark, at the 50% complete mark, at the 75% complete mark, etc.).

At step 708, the quality engine 116 determines whether there is an issue with the network 122. In some examples, the network issue is determined if the network fails to meet the minimum threshold(s) used in step 702. In some examples, higher or lower thresholds may be used to determine whether a network issue has occurred during the measurement.

At step 710, if no network issue is detected, the measurement process continues until it is completed. The quality engine 116 will continue check for network issues while the measurement process is ongoing (steps 706-710). However, if a network issue is detected, the measurement process is paused (step 712). In some examples, the VAQ engine 120 is configured to stop collecting images/videos when the measurement process is paused. Likewise, the VAQ engine 120 will freeze the transmission of frames to the application server 124. In some examples, an alert is displayed to the user 110 via the display 106. The alert may indicate that a network issue was detected and that the measurement process has been paused. In some examples, the alert includes instructions or suggestions for resolving the network issue (e.g., switching Wi-Fi networks).

At step 714, the quality engine 116 determines whether the network issue has been resolved. In some examples, the quality engine 116 will wait for the user 110 to indicate that the issue has been resolved before checking the status of the network 122. The quality engine 116 may determine that the network issue has been resolved by checking that the minimum (or other) thresholds have been met.

If the network issue has been resolved, then at step 716, the measurement process is resumed and the quality engine 116 continues to check for network issues until the measurement process is completed. In the event the network issue has not been resolved, the quality engine 116 may end the measurement process. In some examples, the quality engine 116 is configured to wait for a predetermined amount of time before ending the measurement process (e.g., 1 sec, 5 sec, 30 sec, etc.). In some examples, the quality engine 116 may prompt the user 110 to approve the ending of the measurement process. In some examples, even if the network issue has been resolved, the quality engine 116 may decide that the measurement process should be restarted to ensure measurement accuracy.

### Safety Check

Some user devices, such as smartphones, incorporate central processing unit (CPU) throttling mechanisms to manage thermal conditions by reducing processor speeds during high-intensity computational tasks. This thermal regulation is crucial to prevent overheating, protect internal components, and ensure device longevity. However, in the context of processing pulsatility signals, which requires real-time processing of high-resolution video frames for accurate blood pressure measurement, CPU throttling presents significant challenges. On lower-end devices, the reduced processing power may prevent the client application 112 from handling all frames in real-time, leading to dropped frames or lag, which compromises measurement accuracy. Furthermore, the intensive use of the camera 104 and processing units generates additional heat, potentially causing discomfort or even skin irritation to users who must maintain fingertip contact with the device 102.

To address these challenges, the safety engine 114 of the client application 112 is configured to implement an initial thermal state assessment at the start of each measurement session. In some examples, the safety engine 114 checks the current thermal state of the device 102 using built-in sensors and APIs provided by the operating system of the user device 102. If the thermal state exceeds a predetermined threshold—indicative of higher than anticipated device temperature—the safety engine 114 proactively blocks the measurement process from initiating (or continuing). In some examples, an error message is displayed to the user 110, explaining that the elevated temperature may affect the accuracy of the measurement and could lead to discomfort. By preventing the initiation of the measurement under unfavorable thermal conditions, the safety engine 114 safeguards both the integrity of the data collected and the user's comfort. Additionally, if at any point during the measurement the device's temperature surpasses a critical threshold, the procedure is immediately aborted, and the user 110 is notified accordingly. This approach ensures that measurements are only conducted when optimal conditions are met.

Beyond the initial thermal check, the safety engine 114 continuously monitors the device's thermal state throughout the measurement session. The safety engine 114 checks for any changes in temperature that might affect performance or user comfort. If the thermal state rises above the acceptable threshold at any time, the safety engine 114 responds promptly by interrupting the measurement. In some examples, an error message is presented to the user 110, detailing the thermal issue and suggesting practical measures to bring the device 102 back to an acceptable temperature range. Recommendations may include closing background applications, removing the device from direct sunlight, or allowing it to cool down before retrying. This real-time responsiveness ensures that the user 110 is protected from potential discomfort and that the measurement data remains reliable.

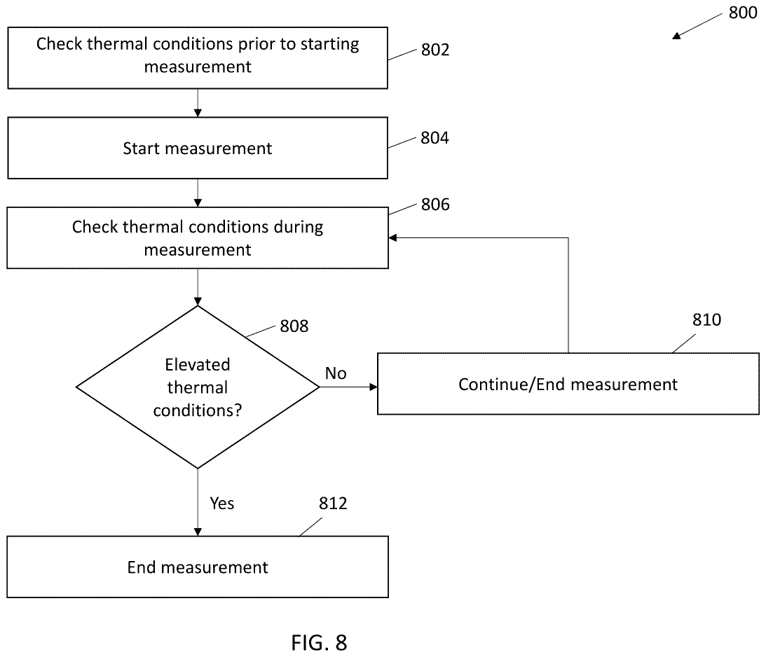

FIG. 8 illustrates a flow diagram of a method 800 for monitoring thermal conditions during the measurement process in accordance with aspects described herein. In some examples, the safety engine 114 of the client application 112 is configured to perform the method 800, at least in part.

At step 802, the safety engine 114 checks the initial thermal conditions of the user device 102 prior to starting the measurement process. In some examples, the safety engine 114 determines whether the thermal conditions are favorable by comparing the thermal conditions (or temperatures) to one or more maximum thermal thresholds. If the thermal conditions exceed the maximum thermal threshold(s), the safety engine 114 prevents the system 100 from proceeding with the measurement process. In some examples, the safety engine 114 alerts the user (e.g., via the display 106) that the thermal conditions are unsafe and/or unsuitable for proceeding with the measurement.

At step 804, in response to a determination that the thermal conditions do not exceed the maximum thermal threshold(s), the safety engine 114 initiates the measurement process. In some examples, the safety engine 114 initiates the measurement process by sending one or more messages to the VAQ engine 120. As described above, the VAQ engine 120 begins to collect frames from the user 110 that are transmitted, via the network 122, to the application server 124 for processing.

At step 806, the safety engine 114 checks the thermal conditions of the user deice 102 during the measurement. In some examples, the safety engine 114 is configured to monitor the thermal conditions continuously. In some examples, the safety engine 114 is configured to check the thermal conditions at periodic intervals (e.g., every second, every 5 seconds, every 30 seconds, etc.). In some examples, the safety engine 114 is configured to check the thermal conditions at specific frame intervals (e.g., every other frame, every 5 frames, every 30 frames, etc.). In some examples, the safety engine 114 is configured to check the network status at certain points in the measurement (e.g., at the 25% complete mark, at the 50% complete mark, at the 75% complete mark, etc.).

At step 808, the safety engine 114 determines whether the thermal conditions have elevated to an unsafe or unsuitable level. In some examples, the safety engine 114 compares the current thermal conditions to the maximum thermal threshold(s) to make this determination.

At step 810, in response to a determination that the thermal conditions do not exceed the maximum thermal threshold(s), the measurement process continues until it is completed. The safety engine 114 continues to check for elevated thermal conditions while the measurement process is ongoing (steps 806-810). However, if elevated thermal conditions are detected (i.e., the thermal conditions exceed the maximum thermal thresholds), the measurement process is ended (step 812). In some examples, an alert is displayed to the user 110 via the display 106. The alert may indicate that the measurement process has ended due to elevated thermal conditions. In some examples, the alert includes instructions or suggestions for resolving the elevated thermal conditions (e.g., closing background applications, waiting a period of time, etc.).

In some examples, to enhance proactive thermal management, the system 100 incorporates an algorithm (or algorithms) to predict potential increases in the thermal state of the user device 102. In some examples, the algorithm is implemented by the AI model 600. By analyzing patterns in device usage, environmental conditions, and historical temperature data, the algorithm can forecast the likelihood of the device 102 overheating during a forthcoming measurement session. If a risk is detected, the safety engine 114 may preemptively warn the user about the possibility of overheating and advises them to delay the measurement. This predictive capability allows users to avoid unsuccessful measurement attempts and reduces the risk of discomfort, thereby improving the overall user experience and efficiency of the client application 112.

In some examples, the safety engine 114 is configured to provide adaptive control of artificial lighting—such as the camera flash or screen illumination—based on the user's readiness to begin the measurement. By optimizing the usage of artificial lighting, the safety engine 114 minimizes unnecessary heat generation, which can contribute to the device 102 overheating. For example, the safety engine 114 may only activate the flash when the user has correctly positioned their finger and is ready to start the measurement. This targeted use of lighting reduces the operational load on the device's hardware, helping to maintain a lower thermal state and conserve battery life.

In addition to controlling lighting based on user readiness, the system 100 may employ the AI model 600 to automatically adjust artificial lighting settings in real-time during the measurement. By analyzing the quality of the video feed and detecting changes in ambient light conditions, the AI model 600 can fine-tune the intensity and duration of the artificial lighting to optimize signal acquisition while minimizing heat production. This dynamic adjustment ensures that the minimum necessary lighting is used to achieve accurate measurements, thereby reducing thermal strain on the user device 102. This AI-driven approach allows the system to balance the need for sufficient illumination against the imperative of maintaining a safe and comfortable device temperature.

By integrating these thermal management strategies—including initial and continuous temperature monitoring, algorithmic thermal prediction, and adaptive lighting control—the system 100 effectively addresses the challenges associated with device overheating during blood pressure measurements. These measures ensure the accuracy of the measurements, protect the user from potential discomfort, and maintain the performance integrity of the device, all of which are critical for the reliable operation of the application.

### Guided Measurements

The client application 112 includes user interface (UI) screens designed to facilitate proper finger placement through visual feedback. In one example, a UI screen displays the raw video captured by the camera 104 in real-time, allowing the user 110 to see exactly how their finger covers the camera (or lens). This direct visual feedback helps users adjust their finger position and pressure to achieve optimal coverage. Alternatively, the client application 112 may present a “virtual” guide generated from the video feed, which simplifies the visual information by highlighting key areas and/or using graphical overlays. This virtual guide can include indicators such as circles or outlines that show the ideal finger placement relative to the camera lens. By providing these visual aids, the client application 112 enhances user understanding and ensures consistent, high-quality measurements.

To assist users in correctly positioning their finger over the camera, the system 100 employs algorithms that analyze the video feed in real-time. In some examples, the algorithms are applied by the quality engine 116 of the client application 112. The algorithms detect features such as the presence of the fingertip, coverage area, and the uniformity of contact. By assessing these factors, the system 100 can determine whether the finger is properly placed. If the placement is suboptimal, the client application 112 provides immediate feedback through on-screen prompts or guidance messages. For instance, if the algorithm detects that the finger is not covering the entire lens, the client application 112 may instruct the user to adjust their finger position. In some examples, the algorithms adapt to variations in finger size, skin tone, and lighting conditions, ensuring that all users receive personalized guidance for optimal finger placement.

In some examples, the system 100 utilizes signal processing methods to evaluate the quality of the pulsatile signal and guide the user accordingly. Such signal processing methods may be performed on the user device 102 (e.g., by the quality engine 116) and/or the application server 124 (e.g., by the processing engine 130). For example, the camera's field of view may be divided into multiple regions of interest (ROIs), which can be specific areas or straps across the image. Within these ROIs, the quality engine 116 calculates parameters such as inter-beat interval (IBI) and signal energy. By analyzing the IBI and energy distribution across different regions, the quality engine 116 can assess the consistency and strength of the pulsatile signal. If certain regions exhibit weaker signals, the quality engine 116 interprets this as an indication of improper finger placement or pressure. By applying logical operations on the distribution of these parameters, the quality engine 116 can provide precise feedback to the user 110, prompting adjustments to improve signal quality.

To enhance the accuracy of finger placement detection, the quality engine 116 may incorporate data from additional sensors available on the user device 102. For instance, if the device 102 is equipped with a pressure-sensitive display or sensor, the quality engine 116 can measure the amount of pressure the user 110 applies when placing their finger over the sensor 104. This information helps ensure that the pressure is neither too light, which may result in poor signal quality, nor too heavy, which could cause discomfort or obscure the lens. By integrating pressure data with visual feedback from the sensor 104, the quality engine 116 provides a more comprehensive assessment of finger placement and guides the user 110 to achieve optimal conditions for measurement.

In some examples, the quality engine 116 leverages a built-in gyroscope and/or and accelerometer of the user device 102 to verify correct device orientation and stability during the measurement process. The gyroscope data allows the quality engine 116 to assess the device's orientation and angle relative to a surface, ensuring that it is held in a position conducive to accurate readings. The accelerometer detects any movement or instability, alerting the user 110 if the user device 102 is shaking or not held steadily. By providing feedback on device positioning, the quality engine 116 helps users maintain the necessary stillness required for precise measurements. Additionally, the quality engine 116 may use audio cues to signal when the device 102 is correctly positioned, further aiding the user 110 without requiring them to look at the display 106.

To enhance user engagement and ensure proper finger placement, the quality engine 116 may incorporate multimodal feedback mechanisms, including audio cues and haptic feedback. Audio cues, such as beeps or voice prompts, inform the user 110 about the correctness of their finger placement and whether the device 102 is stable. Haptic feedback, delivered through vibrations, can signal when optimal finger placement is achieved or when adjustments are needed. For example, a gentle vibration might indicate that the finger is correctly positioned, while a different pattern could alert the user 110 to reposition their finger. By engaging multiple senses, the quality engine 116 provides intuitive and immediate feedback, facilitating a smoother and more effective measurement process.

In some examples, the quality engine 116 employs augmented reality (AR) technology to provide a real-time visual guide overlaid on the user's hand, assisting them in aligning their finger precisely with the camera lens. For example, using the camera 104 and native AR capabilities of the user device 102, the quality engine 116 can detect the user's hand and superimpose graphical elements on the display 106 such as arrows, outlines, or markers that adjust based on hand positioning. This dynamic guide helps users understand how to position their finger in three-dimensional space relative to the device 102, accommodating variations in hand size and holding angles. In some examples, the dynamic guide enhances the user's ability to achieve optimal finger placement quickly and intuitively.

Recognizing that a steady breathing rate can improve measurement conditions, the quality engine 116 may offer assistance to help users regulate their breathing during the procedure. Haptic feedback, such as rhythmic vibrations, can guide the user 110 to inhale and exhale at a steady pace. Similarly, audio cues, like calming tones or guided breathing instructions, support the user 110 in maintaining a consistent breathing pattern. By promoting relaxation and reducing physiological variability, the quality engine 116 helps improve the stability of the pulsatile signal, leading to more accurate blood pressure readings.

In some examples, to ensure that measurements are conducted in a calm and quiet environment, the quality engine 116 utilizes the built-in microphone(s) of the user device 102 to detect ambient sounds. The quality engine 116 analyzes the audio input to identify significant environmental noise, such as loud conversations, traffic, or machinery, which may affect the user's ability to remain still or could introduce artifacts into the measurement. If excessive noise is detected, the quality engine 116 alerts the user and may suggest moving to a quieter location. This proactive approach helps maintain optimal measurement conditions and improves the reliability of the results.

In some examples, the quality engine 116 is configured to integrate data from third-party sources, such as CoreMotion and Apple HealthKit, to provide tailored measurement suggestions based on the user's daily activities. By monitoring factors like physical activity levels, sleep patterns, and periods of rest, the quality engine 116 can identify optimal times for the user 110 to take measurements, such as after a quiet period or during times of low stress. In some examples, the quality engine 116 automatically prompts the user 110 with notifications or reminders to capture health metrics in contexts that are conducive to accurate readings. This personalized approach encourages regular monitoring and helps users incorporate blood pressure measurements into their daily routines effectively.

### Biofeedback

In some examples, the UI of the client application 112 during the measurement process is designed to provide biofeedback. In some examples, the biofeedback varies according to the user's subscription setup. For paying subscribers, the client application 112 may offer a more detailed and engaging experience, displaying comprehensive data visualizations and interactive elements. This may include intricate graphics, advanced metrics, and personalized feedback that enhance user engagement. For free users, the UI may remain intuitive while focusing on essential information to guide them through the measurement without overwhelming detail. This tiered approach ensures that all users receive appropriate feedback while incentivizing upgrades for an enriched experience.

During measurements, the client application 112 may display the live video feed captured by the camera 104 to assist users in correctly positioning their finger. By showing the raw camera input, users can visually confirm that their fingertip adequately covers the lens and adjust as necessary. This real-time visual feedback helps in ensuring optimal finger placement, pressure, and contact, which are crucial for acquiring high-quality pulsatile signals required for accurate blood pressure estimation.

In some examples, the UI includes a progress indicator that informs users about the measurement duration, often segmented into fractions (e.g., ⅓, ⅔, and completion). This timing feedback helps users understand how much time remains, encouraging them to maintain proper finger placement and remain still throughout the process. By providing a clear sense of progression, the system reduces user anxiety and promotes compliance with the measurement protocol. In some examples, the client application 112 displays real-time heart rate (HR) estimates during the measurement to provide immediate biofeedback. To enhance the perception of accuracy and reliability, the client application 112 may apply a three-second averaging filter to smooth out sudden fluctuations in the HR readings. This smoothing technique offers a more stable display, helping users trust the measurement process and remain engaged. The real-time HR visualization also personalizes the experience, allowing users to observe their physiological responses dynamically.

For users with higher entitlements or subscriptions, the client application 112 may provide real-time blood pressure estimates during the measurement. This feature offers an immediate glimpse into their cardiovascular status, enhancing engagement and interest in the process. By observing blood pressure trends in real-time, users can better understand the importance of remaining still and following guidelines, as their actions have a visible impact on the readings. This immediate feedback serves as both an educational tool and a motivator for proper technique.

In some examples, the UI includes signal quality indicators that inform users about the integrity of the data being collected. These indicators may use visual elements such as color codes (e.g., green for good, yellow for moderate, red for poor) or numerical scores to represent signal quality. By providing real-time feedback on signal integrity, users can make necessary adjustments, such as repositioning their finger or reducing movement, to enhance measurement accuracy. This feature empowers users to actively participate in the quality assurance of their readings. In some examples, the client application 112 personalizes the UI by adapting visual elements based on the user's current heart rate. For example, calming colors or animations might be displayed when a higher heart rate is detected, encouraging relaxation. Conversely, more vibrant visuals might accompany a lower heart rate to maintain engagement. This personalization creates a more immersive experience, making the user 110 feel that the application is responsive to their individual physiological state, thus enhancing user satisfaction and adherence to the measurement protocol.

The client application 112 may display the user's PPG waveform in real time, allowing the user 110 to view the unique shape of their pulse wave over time. This personalized visualization allows users to see their cardiovascular activity, providing a tangible connection to their biometric data. By observing the PPG waveform, users can better understand the impact of their actions, such as breathing or movement, on the measurement, leading to improved compliance and technique refinement.

To ensure optimal finger placement, the client application 112 provides specific indications and guidance within the UI. Visual cues such as outlines, hand images, or animations demonstrate where and how to place the finger on the camera lens. If improper placement is detected, the client application 112 prompts the user with corrective instructions, ensuring that the finger fully covers the lens without excessive pressure. This guidance minimizes measurement errors and enhances signal quality by promoting consistent and correct finger positioning.

The features and level of detail provided during the measurement can vary based on user entitlements, such as being an early adopter, subscriber, or frequent user. Users with higher entitlements might access advanced features like detailed analytics, customizable interfaces, or additional biofeedback options. This stratification not only rewards loyal or paying users but also encourages others to engage more deeply with the application to unlock enhanced capabilities, fostering a sense of progression and personalization. As users continue to engage with the client application 112, they may unlock new UI elements or alternative views based on their usage patterns. For example, frequent use might grant access to new visual themes, additional biofeedback metrics, or interactive features. This gamification approach incentivizes regular use and adherence to health monitoring routines. By offering evolving experiences, the client application 112 keeps users motivated and invested in their health journey.

In some examples, the client application 112 is configured to transform the measurement process into a playful mini-game by challenging users to “keep their quality meter stable.” Users receive guidance on finger placement and are rewarded for maintaining optimal conditions. This gamified approach distracts users from the technical aspects, reduces anxiety, and makes the process more enjoyable. By integrating game mechanics, the client application 112 improves user compliance and encourages regular use, ultimately enhancing the accuracy and consistency of the measurements. In some examples, to help users remain still and focused during the measurement, the client application 112 plays simulated white noise through the built-in speaker(s) of the user device 102. This auditory backdrop masks environmental distractions and pro-motes a calm atmosphere conducive to accurate readings. The white noise can help users relax, reduce movement, and maintain the required finger positioning, thereby improving signal quality and the overall effectiveness of the measurement process.

The client application 112 may incorporate other feedback modalities, such as haptic feedback and visual effects like glowing edges of the user device 112. Haptic feedback provides tactile cues, signaling optimal finger placement or alerting users to issues without requiring visual attention. Visual effects can offer subtle, intuitive indicators of measurement progress or signal quality. For example, the device's edges might glow softly when the measurement is proceeding correctly or change color to indicate adjustments are needed. These multisensory feedback options enhance user engagement and aid in maintaining proper measurement conditions.

Acknowledging that emotional state can influence physiological measurements, the client application 112 allows users to select their current mood or energy level before a reading. Based on this input, the client application 112 provides tailored feedback to optimize measurement conditions. For instance, if the user 110 feels stressed, the client application 112 might suggest a brief calming exercise or breathing technique to help stabilize their heart rate. This personalized approach not only improves the accuracy of the readings but also supports users'overall well-being.

In some examples, to ensure consistent and accurate measurements, the client application 112 presents an interactive pre-measurement checklist before each session. The checklist reminds users to adopt optimal conditions, such as sitting comfortably, relaxing, avoiding conversation, and minimizing movement. By establishing a ritualistic preparation routine, the application helps users create a consistent environment for each measurement. This practice enhances the reliability of results and reinforces positive habits in health monitoring.

By incorporating these features into the measurement process, the client application 112 provides comprehensive biofeedback to users, enhancing engagement, accuracy, and overall user experience. These innovations contribute to the effectiveness of the blood pressure measurement system and support users in managing their health proactively.

### Quality Checks

During the measurement phase, the system 100 employs various frame compression methods to efficiently process the RGB video captured by the user device 102 (i.e., the VAQ engine 120). The compression methods may be performed on the user device 102 (e.g., by the compression engine 118) and/or the application server 124 (e.g., by the processing engine 130). In some examples, the quality engine 116 transforms the video frames into multiple PPG signals by segmenting the image into N ROIs. Each ROI generates a separate PPG signal used for quality checks. By analyzing these multiple signals, the quality engine 116 can identify inconsistencies or artifacts in real time, ensuring that only high-quality data is used for blood pressure estimation. This approach optimizes computational resources while maintaining measurement accuracy.

The quality engine 116 calculates several quality indexes in real time, such as signal-to-noise ratio, pulse amplitude, and consistency across PPG signals. If these indexes fall below predefined thresholds, indicating poor signal quality, the quality engine 116 may abort the measurement to prevent inaccurate results. Users are immediately notified with actionable feedback to correct issues like finger placement or excessive movement. This proactive quality control ensures the reliability of the measurements and saves users time by preventing futile attempts. To enhance user experience and signal quality, the quality engine 116 processes optical signals to check parameters such as blurring, deformation patterns, color intensity, and lens distortion. By evaluating these factors, the quality engine 116 can detect issues like unstable finger positioning, improper pressure, or environmental lighting problems. The quality engine 116 provides immediate feedback to the user 110, suggesting adjustments to improve signal acquisition. This real-time analysis and guidance helps users achieve optimal conditions for accurate measurements, improving overall satisfaction.

In some examples, the quality engine 116 optimizes the recording configuration by adjusting flashlight settings—such as illumination levels—and camera focus points based on the processed optical signal. On devices that support these adjustments, the client application 112 can fine-tune these settings to enhance signal clarity. For example, increasing illumination in low-light conditions or adjusting focus to accommodate different finger sizes. This dynamic optimization can also be applied in subsequent measurement phases, ensuring consistent quality across all measurement sessions.

To maintain high-quality pulsatility signals during the measurement phases, the system 100 implements a process of closing biofeedback loops with the user. This interactive approach involves continuous monitoring of the signal quality and providing real-time feedback to the user, enabling them to make immediate adjustments. By engaging the user through these feedback mechanisms, the system 100 ensures that the measurement conditions remain optimal, thereby enhancing the accuracy and reliability of the blood pressure readings. In some examples, the biofeedback process is performed by the quality engine 116 of the client application 112 and/or the processing engine 130 of the application server 124.

In some examples, the biofeedback process includes at least a first loop and a second loop, each targeting different aspects of signal evaluation. In some examples, the first loop corresponds to a signal evaluation that is performed at a first time period after initiating the sensor 104. In some examples, the first time period is the first full heartbeat of the user 110 during the measurement. Likewise, the second loop corresponds to a signal evaluation that is performed at a second time period after initiating the sensor 104. In some examples, the second time period is half of the total signal collection time (or half of the measurement time). In some examples, the second time period is a third of the total signal collection time (or a third of the measurement time).

The first loop focuses on evaluating the quality of the pulsatility signal over short intervals. The quality engine 116 continuously analyzes key parameters such as signal amplitude, consistency, and noise levels. If the signal quality falls below a predefined threshold within this short-term window, the quality engine 116 is notified and promptly prompts the user 110 to react. Notifications may include instructions to adjust finger placement, modify pressure on the camera lens, or reduce movement. By addressing immediate issues as they arise, the first loop enables the user 110 to correct transient problems quickly, ensuring that the measurement proceeds with optimal signal quality.

The second loop assesses the signal quality over a mid-term period, considering a longer segment of the measurement phase (e.g., longer than the first loop periods). If the pulsatility signal consistently fails to meet the required quality standards during this interval, the quality engine 116 may decide to reinitiate the measurement (or recording). This action prevents the continuation of a flawed measurement that could lead to inaccurate results. By restarting the process, the quality engine 116 provides the user 110 with an opportunity to correct any persistent issues, such as environmental interferences or sustained improper finger positioning. The second loop thus serves as a safeguard against prolonged degradation in signal quality.

Beyond the first and second loops, the system 100 may incorporate additional biofeedback mechanisms to further enhance measurement quality. For example, the biofeedback process may include a third loop, a fourth loop, a fifth loop, and so on. In some examples, a third loop evaluates signal quality over the entire duration of the measurement. If cumulative issues are detected, the quality engine 116 may provide comprehensive feedback at the end of the session, offering suggestions for future improvements. Likewise, a fourth loop may analyze data across multiple measurement sessions. The quality engine 116 identifies recurring patterns that affect signal quality and offers personalized advice to the user 110, such as adjusting measurement times or environmental settings. For a fifth loop, the quality engine 116 (or processing engine 130) may dynamically adjust quality thresholds based on user-specific factors, such as skin type or heart rate variability. This personalized approach ensures that feedback remains relevant and effective for different users.

Implementing these biofeedback loops is crucial for preventing user frustration. Without timely feedback, users might unknowingly continue with poor-quality measurements, leading to inaccurate results and wasted time. By promptly alerting the user 110 to issues and providing clear guidance on how to resolve them, the system enhances the user experience. This proactive approach keeps users engaged and confident in the measurement process, reducing the likelihood of abandonment due to repeated failures or unclear instructions.

### Video Compression

The compression of video sequences into pulsatility signals is performed locally on the user device 102 by the compression engine 118. By executing this process on the device 102 itself, the system 100 leverages the device's computational resources to handle data-intensive tasks without the need to transmit large volumes of raw video data over the network 122. Local processing enhances data privacy by keeping sensitive biometric information on the user device 102 during the initial transformation. It also reduces dependency on network connectivity, ensuring that the system 100 can function effectively even in areas with limited or unreliable internet access.

The compression process transforms a sequence of images—each containing several hundreds of thousands to millions of pixels—into one or a series of pulsatility signals that represent the arterial pulsatility of the user's fingertip. This transformation involves extracting vital physiological information from the video frames by analyzing subtle changes in pixel intensities caused by blood flow variations. The resultant pulsatility signals are time-series data that capture the rhythmic patterns of the user's heartbeat, effectively condensing the rich visual data into a meaningful and compact form suitable for further analysis. The extracted pulsatility signals, or in some cases the entire compressed video, serve as input for predictive models of the model engine 126 designed to infer the user's blood pressure values. These model engine 126 utilizes advanced algorithms, such as ML techniques or signal processing methods, to analyze the pulsatility data and estimate systolic and diastolic blood pressure values. By feeding the models with high-quality signals derived directly from the user's physiological data, the system 100 ensures personalized and accurate blood pressure measurements without the need for calibration or external devices.

Compressing the video sequences into pulsatility signals locally (i.e., on the user device 102) significantly reduces both transmission latencies and computational latencies. By decreasing the amount of data that needs to be sent to the application server 124 for processing, the system 100 minimizes network bandwidth usage and accelerates data transfer speeds. This efficiency results in faster delivery of blood pressure results to the user 110, enhancing the overall responsiveness of the client application 112. Additionally, reducing the data volume lowers the computational burden on both the user device 102 and the application server 124, enabling quicker processing times and conserving device resources.

The compression may be achieved using various signal processing methods. In some examples, the pixel values within designated ROIs across successive video frames are averaged to generate corresponding PPG signals. In some examples, color channel analysis is used, where the mean intensity of red, green, and blue channels is calculated over time to extract pulsatile information. Advanced methods like Principal Component Analysis (PCA) or Independent Component Analysis (ICA) may also be applied to reduce dimensionality and isolate the pulsatility signals from noise. These techniques effectively condense the rich video data into the essential signals that retain the critical information necessary for accurate blood pressure estimation.

In the process of transforming video data into pulsatility signals, some steps may be lossless, preserving all relevant information, while others may be lossy, intentionally discarding certain details to achieve greater compression. Lossless steps include operations like precise mathematical transformations or encoding schemes that maintain data integrity. Lossy steps might involve filtering out high-frequency noise or redundant data that do not contribute significantly to the accuracy of blood pressure estimation. By balancing lossless and lossy processing, the system 100 optimizes the size of the data for efficient transmission and computation without compromising the quality of the pulsatility signals used by the predictive models.

By implementing these strategies, the compression engine 118 effectively compresses large video sequences into manageable pulsatility signals, facilitating rapid and accurate blood pressure measurements. The combination of local processing, efficient compression techniques, and thoughtful consideration of data preservation ensures that the system 100 delivers reliable results while maintaining user privacy and optimizing performance.

In addition to transmitting the compressed pulsatility signals used for immediate blood pressure calculation, the client application 112 may operate in the background to send a less compressed version of the data to the application server 124. This background data transmission occurs asynchronously and independently of the primary measurement flow, ensuring that it does not interfere with the user's experience or delay the delivery of blood pressure results. By handling this process in the background, the system 100 maintains optimal usability while still collecting valuable data for further analysis.

The less-compressed data sent in the background is not utilized for calculating the blood pressure values presented to the user 110 during the current measurement session. Instead, the model engine 126 uses the less-compressed data for reinforcement learning of the BP predictive models and/or the training of foundational models. By providing richer and more detailed datasets, the system 100 enables continuous improvement of algorithm accuracy, robustness, and adaptability. This ongoing learning process allows the blood pressure models to better capture subtle physiological variations and environmental factors, ultimately enhancing the precision of future blood pressure estimations for all users.

Performing the transmission of less-compressed data in the background and independently of the main data flow offers several usability advantages. In some examples, the compressed data used for calculating the blood pressure values is smaller in size, allowing it to be transmitted to the application server 124 more quickly. This results in reduced transfer times, enabling users to receive their BP readings promptly. Additionally, the smaller data size minimizes data transfer costs, which is particularly beneficial for users with metered or limited network services. In some examples, the compressed data is transmitted to the application server 124 and the less-compressed data is transmitted to a different server. By sending only the compressed data to the first-line production server(s) (e.g., the application server 124), the system reduces the computational load and bandwidth requirements on these servers. This optimization allows for reduced dimensioning of server resources, enhancing scalability and efficiency in processing user requests.

The separation of data transmission processes ensures that the user's interaction with the client application 112 remains smooth and uninterrupted. The background transfer of less-compressed data does not impact the performance or the responsiveness of the client application 112, thereby maintaining a high level of user satisfaction.

In some examples, the less-compressed data includes the full video captured during the measurement, containing all frames and high-resolution pixel data, providing comprehensive information valuable for in-depth analysis and model training. The less-compressed data may include additional ROIs beyond the ROIs used in the initial compression for immediate blood pressure calculation. This additional spatial information enhances the diversity and richness of the dataset, facilitating improved model learning. In some examples, the client application 112 may send less-compressed versions of the pulsatility signals that retain more of the original data's nuances. This allows the models to learn from finer details in the signal patterns, improving their predictive capabilities.

In some examples, the use of compressed data for real-time blood pressure calculation ensures quick results and efficient use of network resources. The background transmission of richer data supports the ongoing development of the models, leading to progressively better blood pressure estimations. By handling additional data transmission in the background, the system 100 preserves the usability and responsiveness of the client application 112, providing a seamless experience for the user 110. By transmitting these forms of less-compressed (or uncompressed) data in the background, the system 100 accumulates a wealth of information that contributes to the continuous refinement of the predictive models. This strategy ensures that the models evolve over time to become more accurate and reliable, ultimately benefiting all users with improved blood pressure measurement performance. The background sending of less-compressed data is a strategic feature of the system 100 that balances immediate user needs with long-term model enhancement.

### Predictive Models

To optimize the performance and maintain the integrity of the blood pressure measurement system 100, the predictive models are run in the cloud (e.g., on the model engine 126 of the application server 124) rather than on the user device 102. This cloud-based approach offers several significant advantages that enhance the system's efficiency, scalability, and security. The predictive models used to infer blood pressure from pulsatility signals are often highly complex and may consist of extensive neural networks or advanced ML algorithms requiring substantial computational resources. These models can be very large in size, potentially exceeding the storage and processing capabilities of standard user devices. By running the models in the cloud, the system 100 leverages powerful server infrastructures capable of handling large-scale computations efficiently. This approach ensures that the user device 102 is not burdened with heavy processing tasks, allowing for faster application performance and preserving battery life. It also enables the utilization of more sophisticated models that can improve accuracy without being constrained by the limitations of local hardware.

Cloud-based models allow for continuous updates and improvements without necessitating user intervention or frequent application updates. As new data is collected and ML techniques advance, the models can evolve over time to become more accurate and reliable. Running the models in the cloud facilitates seamless integration of updates, ensuring that all users benefit from the latest enhancements immediately. This dynamic adaptability enables maintaining the system's relevance and effectiveness in the rapidly evolving field of digital health technologies. It simplifies maintenance and version control by centralizing the models, avoiding discrepancies that could arise from multiple versions distributed across various user devices.

By keeping the predictive model(s) on secure cloud servers, the system 100 protects its sensitive algorithms from unauthorized access. Running the model locally would expose it to potential security risks. Cloud deployment ensures that core information remains confidential and secure behind robust cybersecurity measures. This approach ensures compliance with legal and regulatory standards related to data protection. It also builds user trust by demonstrating a commitment to protecting sensitive components of the technology.

While it may be preferred to run the predictive models in the cloud (e.g., on the application server 124), it should be appreciated that the predictive models may be run on the user device 102. In this alternative scenario, the model(s) may be optimized for mobile platforms, ensuring it is lightweight enough to function within the computational constraints of the user device 102. Running the model locally can offer benefits such as reduced latency, as there is no need to transmit data to the cloud and wait for a response. It also enhances privacy by keeping all data processing confined to the user device 102, which may be preferred in situations with limited network connectivity or where users have heightened privacy concerns. This embodiment provides flexibility and broadens the applicability of the system 100 across different user preferences and technological environments. By strategically choosing between cloud-based and local model execution, the system 100 balances performance, adaptability, security, and user convenience. This flexibility ensures that the blood pressure measurement system 100 remains effective and accessible, delivering accurate results while accommodating the evolving needs of users and advancements in technology.

### Data Presentation

For first-time users, the client application 112 may present an introductory message that frames the initial blood pressure reading as a starting point rather than a definitive baseline. This message acknowledges that the initial measurement may be influenced by factors such as nervousness, recent physical activity, or unfamiliarity with the measurement process. By setting appropriate expectations, the client application 112 reduces potential anxiety and helps users understand that subsequent readings will provide a more accurate reflection of their typical blood pressure levels. To assist new users in establishing an accurate baseline, the client application 112 may recommend a personalized measurement schedule. The schedule prompts users to take readings consistently, such as at the same time on specific days each week. This consistency minimizes variability due to circadian rhythms and daily activities, leading to more reliable trend analysis. The client application 112 may provide reminders or notifications to encourage adherence to the suggested schedule, fostering regular monitoring habits and enhancing the accuracy of future readings.

For users with an established baseline, each new reading may be accompanied by contextual feedback that compares the current measurement to their typical range. If a reading falls within their normal range, the client application 112 offers positive reinforcement, acknowledging the user's efforts in maintaining healthy blood pressure levels. Conversely, if a reading deviates from the usual range, the client application 112 prompts the user 110 to consider potential contributing factors, such as recent stress, dietary changes, or physical activity. This personalized feedback enhances user engagement and promotes self-awareness of how lifestyle factors impact their blood pressure.

In some examples, the client application 112 allows users to select from a customizable list of common factors that may affect blood pressure, enabling easy tracking and deeper trend analysis. For example, the factors may include physical activity, caffeine intake, alcohol consumption, high-sodium meal consumption, medication intake, sleep quality, stress levels, hydration level, temperature, measurement posture, meal timing, nicotine use, recent pain or discomfort, hormonal fluctuations, and other relevant factors. By building a quick-access list of frequently selected factors, users can efficiently log relevant influences for each measurement. This tailored tracking empowers the system 100 to generate more precise insights into how specific lifestyle elements affect individual blood pressure readings over time. In some examples, the user-specific factors are organized and managed by the user engine 128 and stored in the user data database 132b.

To strengthen self-awareness and reinforce the connection between daily habits and blood pressure, the client application 112 may prompt users to evaluate their recent lifestyle choices before displaying their reading. Users are encouraged to reflect on factors such as stress levels, physical activity, caffeine intake, or sleep quality. This pre-reading reflection promotes mindfulness and helps users recognize potential influences on their blood pressure, fostering proactive health management. Likewise, after each measurement, the client application 112 may provide a recap prompt that encourages users to consider how their recent habits may have impacted the reading. For example, it might ask, “You noted feeling stressed today. Did taking a few deep breaths help with this reading?” This post-reading reflection reinforces the effect of lifestyle adjustments and helps users internalize how their actions influence their results, promoting sustained behavioral changes. When a measurement falls outside the user's typical range, the client application 112 may offer tailored advice to address the deviation. For instance, it may suggest, “This reading is higher than usual. Consider a quick breathing exercise, then try measuring again once you feel calm.” These actionable tips provide immediate steps the user can take to improve future readings, supporting healthier measurement habits and encouraging active participation in managing their blood pressure.

As users consistently log common influences over time, the client application 112 adapts by prioritizing these factors in the UI. This makes it quicker for users to select and track recurring influences, enhancing the efficiency of data entry. The system 100 utilizes this information to provide more tailored insights into how specific lifestyle elements affect individual readings. Over time, users receive personalized recommendations and trend analyses that support their health goals and enhance the effectiveness of the monitoring process.

To motivate users, the client application 112 may incorporate gamification elements such as achievement badges. Users earn badges for reaching milestones like consistent measurement taking (“Consistent Tracker”), maintaining readings within their target range (“Accurate Analyzer”), or completing their initial measurement (“First Step”). These badges may be displayed in the user's profile, serving as a motivational record and fostering a sense of accomplishment. In some examples, the client application 112 sets specific targets, such as maintaining a seven-day streak of measurements or achieving consistent readings within a healthy range over consecutive days. Achieving these goals unlocks unique rewards or recognition within the client application 112, reinforcing regular monitoring and building sustained engagement. This goal-oriented approach encourages users to integrate blood pressure monitoring into their daily routines. In some examples, a visual progress bar within the client application 112 shows users how close they are to achieving their next goal or milestone. This immediate feedback provides motivation and encourages users to maintain healthy habits to reach their objectives. By making progress tangible and visually engaging, the system 100 enhances user commitment to regular monitoring.

To celebrate achievements and share progress, users can create and share snapshot cards directly after measurements. These cards display the user's latest values, earned badges, or overall progress, and can be shared with friends and family. This feature enables users to involve their support network in their health journey, whether to proudly announce progress or to encourage loved ones to stay healthy together, promoting accountability and social motivation. In some examples, the client application 112 facilitates social engagement by allowing users to set up groups with friends or family members. Within these groups, users can participate in health-based challenges, such as maintaining a weekly average within a healthy range or tracking measurements daily for a month. Group progress is displayed visually, and customizable rewards are provided for achieving collective goals. This collaborative approach fosters a supportive environment that promotes healthy competition and accountability. Likewise, users can create or join small health groups, known as “Health Circles,” where they can share snapshots, send encouraging messages, or celebrate health milestones together. These health circles provide a platform for social motivation and accountability, enabling users to engage with others who have similar health goals. By building a sense of community, the application enhances user commitment and provides emotional support throughout their health journey.

In some examples, the client application 112 provides regular insights that summarize the user's blood pressure trends over time, highlighting periods of stability or noticeable changes. For example, a summary might state, “Your blood pressure was stable this week—keep up the great work!” These overviews give users a broad perspective on their health progress, helping them recognize patterns and identify areas for improvement. Presenting information in an accessible and encouraging manner supports sustained engagement and informed decision-making. Based on observed data and user behavior, the application may display personalized insight cards with specific, actionable recommendations. For instance, it might highlight patterns such as, “Your readings tend to be lower in the evenings. Consider establishing an evening relaxation routine.” These tailored suggestions help users understand how their lifestyle choices impact their blood pressure and provide practical steps to optimize their health. Delivering insights that are directly relevant to the user's experiences enhances the value of the monitoring process and promotes positive behavioral changes.

By integrating these features, the system presents blood pressure values to users in a manner that is not only informative but also engaging and supportive. The combination of personalized insights, gamification, social engagement, and adaptive recommendations encourages consistent monitoring, fosters healthier habits, and empowers users to take an active role in managing their cardiovascular health. This comprehensive approach enhances user satisfaction and contributes to the overall effectiveness of the blood pressure measurement system.

### Integration

While the above description of the system 100 includes a single user device 102 for the user 110, it should be appreciated that the system 100 can include multiple devices per user. For example, the user 110 may be associated with multiple devices (e.g., a smartphone, a smartwatch, a tablet, etc.) that are compatible with the system 100. In such examples, the user 110 may access their user account and measurements on different devices via the client application 112. The user's account may keep track of the devices associated with the user 110. In some examples, the user 110 can view previous measurements and record new measurements on each of their compatible device. In some examples, the user 110 can use the client application 112 to view measurements on a first device (e.g., a smartphone) that are being recorded, at least partially, using a second device (e.g., a wristband device).

In some examples, the client application 112 is configured to recommend new devices or sensors for the user 110 to purchase. Based on the blood pressure values that are presented to the user 110, the client application 112 may assist the user 110 in ordering a second device (or sensor) that is capable of receiving signals from the user 110 for future blood pressure measurements. For example, if the user's blood pressure value is high, and therefore warrants frequent monitoring, the client application 112 may recommend a wearable device that enables the user 110 to collect frequent measurements. Likewise, the client application 112 may recommend different subscription tiers based on the user's blood pressure values and other lifestyle factors. In both examples, the client application 112 may place the order for the user 110 and collect payment to facilitate the purchase.

In some examples, a first sensor (or device) is used as a spot-check sensor to screen, diagnose, detect, or identify a health condition of the user 110. In such examples, the second sensor (or device) is used to manage the health condition. For example, the second sensor may be a long-term wearable sensor, or a frequent-use wearable sensor, that monitors blood pressure (or other metrics) in accordance with a management or treatment plan for the health condition. The first sensor may be a sensor that is available to everyone (e.g., a smartphone), allowing the screening to be done at large scale across the population. The second sensor can be obtained (or ordered) via the client application 112, providing easy and frequent passive readings of blood pressure with no need for the user 110 to do any particular actions to obtain measurements. Use of the second sensor engages the user in the management of the health condition for the long term, and ultimately leads to improved health outcomes.

In one example, the first sensor identifies that the user 110 has high blood pressure and the second sensor is used by the user 110 to manage hypertension. In another example, the first sensor identifies that the user 110 has low blood pressure and the second sensor is used by the user 110 to manage hypotension. In one example, the first sensor identifies that the user has abnormal blood pressure and the second sensor is used by the user 110 to manage blood pressure. In another example, the first sensor identifies that the user 110 has abnormal glucose levels in their blood and the second sensor is used by the user 110 to manage diabetes. It should be appreciated that the health conditions described above are examples and are not intended to be limiting.

In some examples, the first sensor is used for a first purpose and the second sensor is used for a second purpose. The purposes of the first and second sensors may be the same or different. For example, the first sensor may identify that a user has blood pressure or heart rate readings that are abnormal (e.g., high, low, or discrepant from prior readings) and the second sensor may be used to confirm or refute the readings given by the first sensor. In such examples, no treatment or management is initiated, but the second sensor is used to give continuous (or periodic) data which gives the user 110 clarity on their blood pressure or heart rate assessment.

In some examples, the first sensor is used by a single user or a group of users (e.g., mass population screening) by a clinical practice, payer, or government organization to identify people who might be “high-risk” based on the readings given by the first sensor. High-risk may be defined by elevated blood pressure readings from the first sensor. The users who are identified as high risk are given the second sensor to confirm and manage the blood pressure by the clinical practice, payer, or government. The benefits of such use may include better health outcomes, lower population blood pressure values, and lower cost to the payer system.

In some examples, if the first sensor identifies possible higher readings, the user can obtain the second sensor through the client application 112 and initiate a digital blood pressure management program using the data collected from the second sensor. In some examples, the first sensor is used to make a diagnosis of hypertension and the second sensor is used by the user 110 (or physician) to titrate medications, confirm compliance, and success of treatment, as well as monitor the user's blood pressure over the long term.

In some cases, medical professionals use two spot-check readings of blood pressure to make a diagnosis. This can lead to inaccurate diagnoses due to discrepancies between the two readings. As such, the accuracy of diagnoses can be improved by using the first sensor to screen for possible conditions (e.g., hypertension) and the second sensor to make a diagnosis based on continuous (or periodic) data. In such examples, accuracy is improved by shifting the diagnosis from episodic monitoring to requiring continuous (or periodic) data from the second sensor.

In some examples, the system 100 is configured to use the first sensor differently once the second sensor has been added. For example, the display 106 of the user device 102 may be used differently after user 110 adds a second sensor or device (e.g., a wearable device) to the system 100.

In some examples, the addition of the second sensor causes the system 100 to update the account of the user 110. For example, the system 100 may transition the account of the user 110 from association with the first sensor to association with the second sensor. In some examples, the user account is updated to reflect that the first sensor is a primary sensor and the second sensor is a secondary sensor, or vice versa. In some examples, transitioning of the account occurs without a need for the user 110 to create a new account or download a separate application. In some examples, the transition includes integrating historical data collected by the first sensor (e.g., readings, measurements, diagnoses, conditions, preferences, etc.) with data collected by the second sensor (e.g., continuous or periodic measurements). In some examples, the transition includes integrating morphological data collected by the first sensor with data collected by the second sensor. In some examples, the transition includes integrating personalized settings associated with the first sensor with personalized settings associated with the second sensor. In some examples, the transition includes integrating a user health profile associated with the first sensor with a user health profile associated with the second sensor.

### Methods of Operation

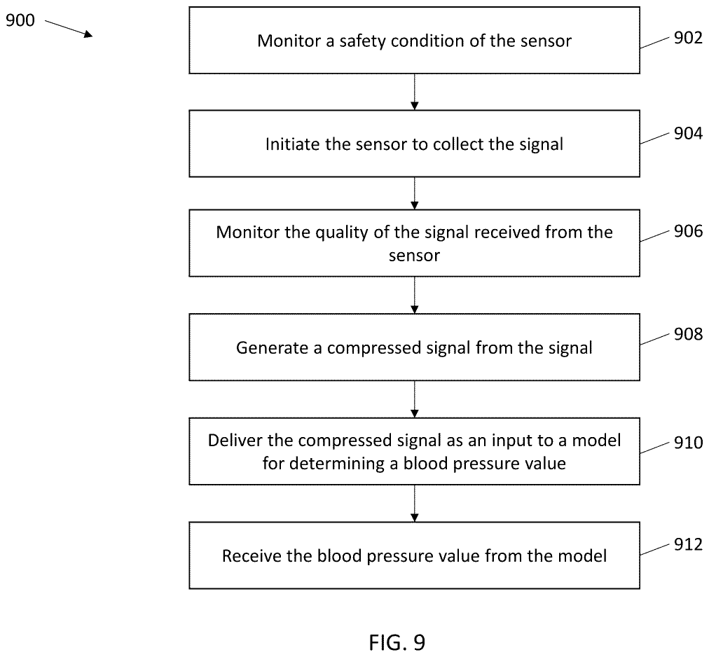

FIG. 9 illustrates a flow diagram of a method 900 for determining a blood pressure value of a user in accordance with aspects described herein. In some examples, the system 100 is configured to perform the method 900.

At step 902, the system 100 monitors a safety condition of the sensor 104. In some examples, the sensor 104 includes, or is included in, a video acquisition system. In some examples, the video acquisition system is embedded within a smartphone (i.e., the user device 102). In some examples, the safety engine 114 of the client application 112 monitors the safety condition of the sensor 104. In some examples, the safety condition is one or more thermal conditions of the client device 104. In some examples, the monitoring the safety condition includes determining a temperature of the sensor 104. In some examples, the safety engine 114 prevents operation of the sensor 104 upon determining that the temperature of the sensor 104 exceeds a predetermined temperature threshold. In some examples, the safety condition of the sensor 104 is monitored in accordance with the method 800 of FIG. 8.

At step 904, the system 100 initiates the sensor 104 to collect the signal. In some examples, the VAQ engine 120 of the client application 112 is configured to initiate the sensor 104 and operate the sensor 104 to collect the signal. In some examples, the signal is a pulsatility or pulsatile signal.

At step 906, the system 100 monitors the quality of the signal received from the sensor 104. In some examples, the quality engine 116 of the client application 112 monitors the quality of the signal. In some examples, monitoring the quality of the signal received from the sensor 104 includes (i) evaluating the quality of the signal at a first time period after initiating the sensor 104 and (ii) evaluating the quality of the signal 104 at a second time period after initiating the sensor 104. In some examples, the quality engine 116 prompts the user 110 and restart collection of the signal upon determining that the quality of the signal is below a predetermined quality threshold.

At step 908, the system 100 generates a compressed signal from the signal. In some examples, the compression engine 118 of the client application 112 is configured to generate the compressed signal.

At step 910, the system 100 delivers the compressed signal as an input to a model for determining a blood pressure value. In some examples, the compressed signal is transmitted to a remote server storing the model (e.g., the application server 124). In some examples, the compressed signal is delivered from the client application 112 of the user device 102 to the model engine 126 of the application server 124. In some examples, the compressed signal is delivered to the processing engine 130 of the application server for pre-processing and conditioning before it its delivered to the model engine 126. In some examples, the client application 112 is configured to transmit the signal in a format larger than the compressed signal to a remote storage location (e.g., the signal data database 132c of the application server 124).

At step 912, the system 100 receives the blood pressure value from the model. In some examples, the blood pressure value is transmitted from the model engine 126 of the application server 124 to the client application 112 of the user device 102. In some examples, the client application 112 presents information related to the blood pressure value to the user 110.

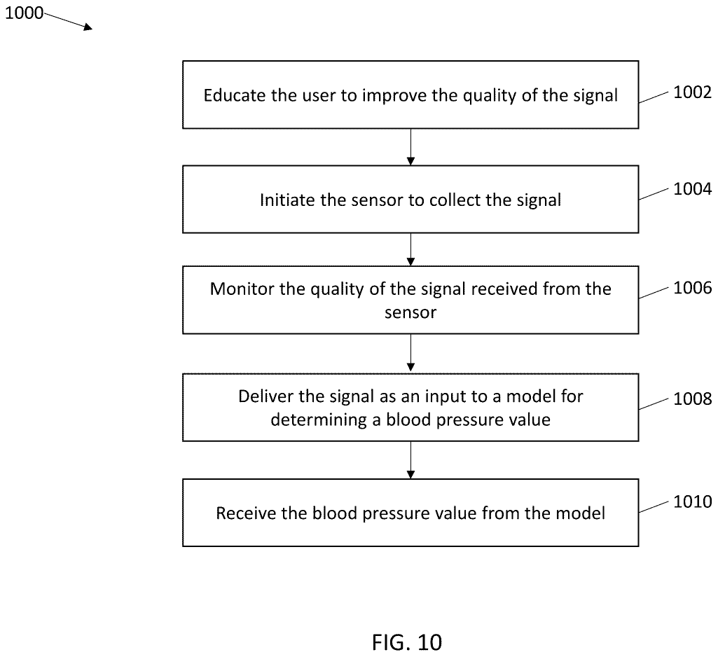

FIG. 10 illustrates a flow diagram of a method 1000 for monitoring blood pressure in accordance with aspects described herein. In some examples, the system 100 is configured to perform the method 1000.

At step 1002, the system 100 educates the user 110 to improve the quality of the signal. In some examples, the quality engine 116 of the client application 112 is configured to present content that instructs or guides the user 110 to improve signal quality. In some examples, the quality engine 116 presents information to the user 110 regarding sensor settings. In some examples, the quality engine 116 presents information to the user 110 regarding placement of the sensor 104.

At step 1004, the system 100 initiates the sensor 104 to collect the signal. In some examples, the VAQ engine 120 of the client application 112 is configured to initiate the sensor 104 and operate the sensor 104 to collect the signal. In some examples, the signal is a pulsatility or pulsatile signal.

At step 1006, the system 100 monitors the quality of the signal received from the sensor 104. In some examples, the quality engine 116 of the client application 112 monitors the quality of the signal. In some examples, monitoring the quality of the signal received from the sensor 104 includes (i) evaluating the quality of the signal at a first time period after initiating the sensor 104 and (ii) evaluating the quality of the signal 104 at a second time period after initiating the sensor 104. In some examples, the first time period occurs during a time period of a first full heartbeat of the user 110 and the second time period occurs during a first half of the total signal collection time period. In some examples, the quality engine 116 prompts the user 110 and restart collection of the signal upon determining that the quality of the signal is below a predetermined quality threshold.

At step 1008, the system 100 delivers the signal as an input to a model for determining a blood pressure value. In some examples, the signal is transmitted to a remote server storing the model (e.g., the application server 124). In some examples, the signal is delivered from the client application 112 of the user device 102 to the model engine 126 of the application server 124. In some examples, the signal is delivered to the processing engine 130 of the application server for pre-processing and conditioning before it its delivered to the model engine 126.

At step 1010, the system 100 receives the blood pressure value from the model. In some examples, the blood pressure value is transmitted from the model engine 126 of the application server 124 to the client application 112 of the user device 102. In some examples, the client application 112 presents information related to the blood pressure value to the user 110.

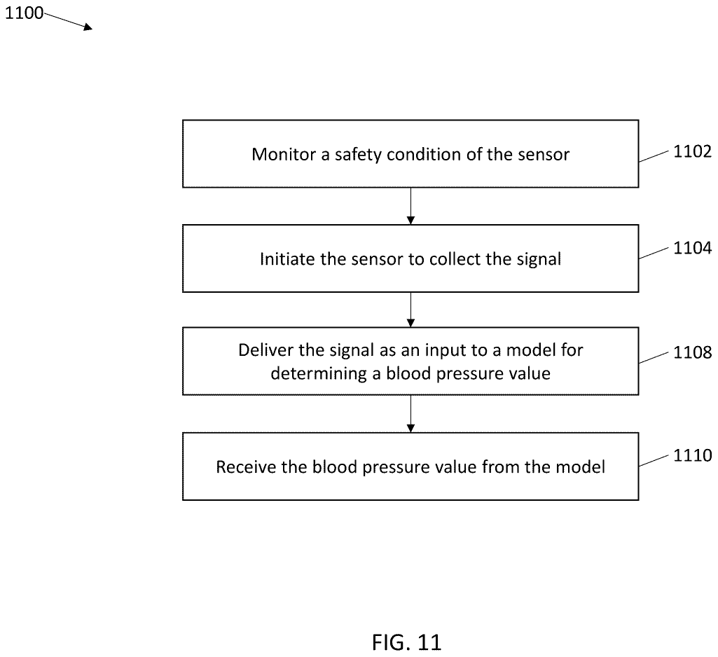

FIG. 11 illustrates a flow diagram of a method 1100 for determining a blood pressure value of a user in accordance with aspects described herein. In some examples, the system 100 is configured to perform the method 1100.

At step 1102, the system 100 monitors a safety condition of the sensor 104. In some examples, the sensor 104 includes, or is included in, a video acquisition system. In some examples, the video acquisition system is embedded within a smartphone (i.e., the user device 102). In some examples, the safety engine 114 of the client application 112 monitors the safety condition of the sensor 104. In some examples, the safety condition is one or more thermal conditions of the client device 104. In some examples, the monitoring the safety condition includes determining a temperature of the sensor 104. In some examples, the safety engine 114 prevents operation of the sensor 104 upon determining that the temperature of the sensor 104 exceeds a predetermined temperature threshold. In some examples, the safety condition of the sensor 104 is monitored in accordance with the method 800 of FIG. 8.

At step 1104, the system 100 initiates the sensor 104 to collect the signal. In some examples, the VAQ engine 120 of the client application 112 is configured to initiate the sensor 104 and operate the sensor 104 to collect the signal. In some examples, the signal is a pulsatility or pulsatile signal.

At step 1106, the system 100 delivers the signal as an input to a model for determining a blood pressure value. In some examples, the signal is transmitted to a remote server storing the model (e.g., the application server 124). In some examples, the signal is delivered from the client application 112 of the user device 102 to the model engine 126 of the application server 124. In some examples, the signal is delivered to the processing engine 130 of the application server for pre-processing and conditioning before it its delivered to the model engine 126.

At step 1110, the system 100 receives the blood pressure value from the model. In some examples, the blood pressure value is transmitted from the model engine 126 of the application server 124 to the client application 112 of the user device 102. In some examples, the client application 112 presents information related to the blood pressure value to the user 110.

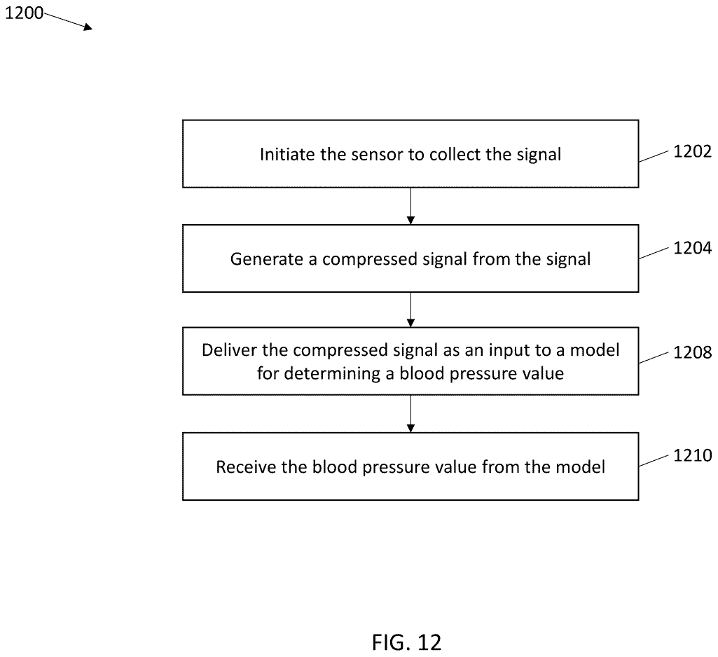

FIG. 12 illustrates a flow diagram of a method 1200 for determining a blood pressure value of a user in accordance with aspects described herein. In some examples, the system 100 is configured to perform the method 1200.

At step 1202, the system 100 initiates the sensor 104 to collect the signal. In some examples, the VAQ engine 120 of the client application 112 initiates the sensor 104 and operate the sensor 104 to collect the signal. In some examples, the signal is a pulsatility or pulsatile signal.

At step 1204, the system 100 generates a compressed signal from the signal. In some examples, the compression engine 118 of the client application 112 generates the compressed signal.

At step 1206, the system 100 delivers the compressed signal as an input to a model for determining a blood pressure value. In some examples, the compressed signal is transmitted to a remote server storing the model (e.g., the application server 124). In some examples, the compressed signal is delivered from the client application 112 of the user device 102 to the model engine 126 of the application server 124. In some examples, the compressed signal is delivered to the processing engine 130 of the application server for pre-processing and conditioning before it its delivered to the model engine 126. In some examples, the client application 112 transmits the signal in a format larger than the compressed signal to a remote storage location (e.g., the signal data database 132c of the application server 124).

At step 1208, the system 100 receives the blood pressure value from the model. In some examples, the blood pressure value is transmitted from the model engine 126 of the application server 124 to the client application 112 of the user device 102. In some examples, the client application 112 presents information related to the blood pressure value to the user 110.

### Hardware and Software Implementations

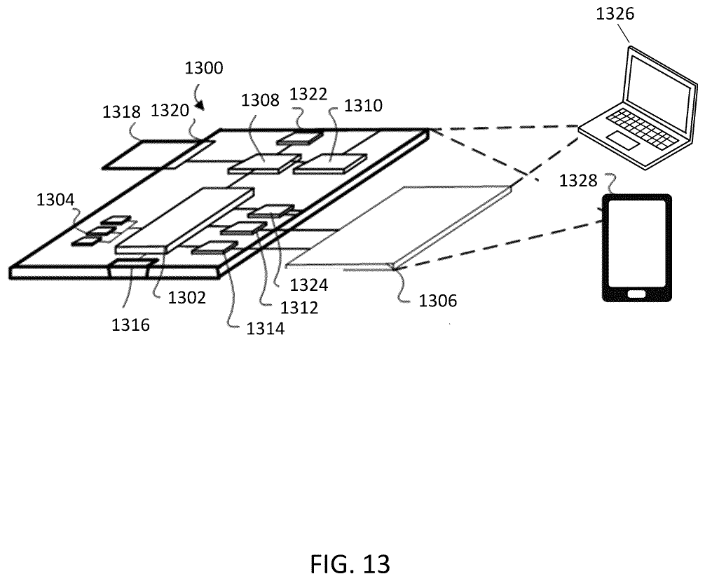

FIG. 13 shows an example of a generic computing device 1300, which may be used with some of the techniques described in this disclosure (e.g., as user device 102 or application server 124). Computing device 1300 includes a processor 1302, memory 1304, an input/output device such as a display 1306, a communication interface 1308, and a transceiver 1310, among other components. The device 1300 may also be provided with a storage device, such as a micro-drive or other device, to provide additional storage. Each of the components 1300, 1302, 1304, 1306, 1308, and 1310, are interconnected using various buses, and several of the components may be mounted on a common motherboard or in other manners as appropriate.

The processor 1302 can execute instructions within the computing device 1300, including instructions stored in the memory 1304. The processor 1302 may be implemented as a chipset of chips that include separate and multiple analog and digital processors. The processor 1302 may provide, for example, for coordination of the other components of the device 1300, such as control of user interfaces, applications run by device 1300, and wireless communication by device 1300.

Processor 1302 may communicate with a user through control interface 1312 and display interface 1314 coupled to a display 1306. The display 1306 may be, for example, a TFT LCD (Thin-Film-Transistor Liquid Crystal Display) or an OLED (Organic Light Emitting Diode) display, or other appropriate display technology. The display interface 1314 may comprise appropriate circuitry for driving the display 1306 to present graphical and other information to a user. The control interface 1312 may receive commands from a user and convert them for submission to the processor 1302. In addition, an external interface 1316 may be provided in communication with processor 1302, so as to enable near area communication of device 1300 with other devices. External interface 1316 may provide, for example, for wired communication in some implementations, or for wireless communication in other implementations, and multiple interfaces may also be used.

The memory 1304 stores information within the computing device 1300. The memory 1304 can be implemented as one or more of a computer-readable medium or media, a volatile memory unit or units, or a non-volatile memory unit or units. Expansion memory 1318 may also be provided and connected to device 1300 through expansion interface 1320, which may include, for example, a SIMM (Single In Line Memory Module) card interface. Such expansion memory 1318 may provide extra storage space for device 1300, or may also store applications or other information for device 1300. Specifically, expansion memory 1318 may include instructions to carry out or supplement the processes described above, and may include secure information also. Thus, for example, expansion memory 1318 may be provided as a security module for device 1300, and may be programmed with instructions that permit secure use of device 1300. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer-or machine-readable medium, such as the memory 1304, expansion memory 1318, memory on processor 1302, or a propagated signal that may be received, for example, over transceiver 1310 or external interface 1316.

Device 1300 may communicate wirelessly through communication interface 1308, which may include digital signal processing circuitry where necessary. Communication interface 1308 may in some cases be a cellular modem. Communication interface 1308 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1310. In addition, short-range communication may occur, such as using a Bluetooth, WiFi, or other such transceiver (not shown). In addition, GPS (Global Positioning System) receiver module 1322 may provide additional navigation- and location-related wireless data to device 1300, which may be used as appropriate by applications running on device 1300.

Device 1300 may also communicate audibly using audio codec 1324, which may receive spoken information from a user and convert it to usable digital information. Audio codec 1324 may likewise generate audible sound for a user, such as through a speaker, e.g., in a handset of device 1300. Such sound may include sound from voice telephone calls, may include recorded sound (e.g., voice messages, music files, etc.) and may also include sound generated by applications operating on device 1300. In some examples, the device 1300 includes a microphone to collect audio (e.g., speech) from a user. Likewise, the device 1300 may include an input to receive a connection from an external microphone.

The computing device 1300 may be implemented in a number of different forms, as shown in FIG. 13. For example, it may be implemented as a computer (e.g., laptop) 1326. It may also be implemented as part of a smartphone 1328, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation.

Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

We also disclose the existing set of claims but with the claim dependency changed for the dependent claims so that they are dependent from any preceding claim.

1-32. (canceled)

33. A system for hypertension screening and monitoring, comprising:
a user device;
a first pulsatility sensor configured to collect, from a user, at least one photoplethysmography (PPG) signal;
a second pulsatility sensor configured to collect, from the user, at least one pulsatility signal; and
a processor electronically coupled to the user device and to the first and second pulsatility sensors, the processor being programmed to perform operations comprising:
processing the at least one PPG signal from the first pulsatility sensor to automatically generate a detection of a health condition of the user, the health condition comprising an identification of a hypertension risk; and
in response to detecting the health condition, automatically generating and causing the user device to display a notification,
wherein the notification comprises a recommendation for the user to use the second pulsatility sensor to obtain the at least one pulsatility signal and process the at least one pulsatility signal to determine at least one blood pressure value.

34. The system of claim 33, wherein the user device comprises at least one of a smartphone, a tablet, or a smartwatch.

35. The system of claim 33, wherein the first pulsatility sensor comprises a noninvasive optical sensor.

36. (canceled)

37. The system of claim 33, wherein the processor is further programmed to perform operations comprising:
facilitating the user obtaining the second pulsatility sensor by collecting payment from the user for the second pulsatility sensor.

38. The system of claim 33, wherein the second pulsatility sensor is of a same type as the first pulsatility sensor.

39. The system of claim 33, wherein the second pulsatility sensor is of a different type than the first pulsatility sensor.

40. (canceled)

41. The system of claim 33, wherein the second pulsatility sensor is comprised in a wearable device.

42. The system of claim 33, wherein the second pulsatility sensor is comprised in a wristband.

43. (canceled)

44. The system of claim 33, wherein the processor is further programmed to perform operations comprising:
transitioning an account of the user from association with the first pulsatility sensor to association with the second pulsatility sensor.

45. The system of claim 44, wherein transitioning the account occurs without a need for the user to create a new account or download a separate application.

46. The system of claim 44, wherein transitioning the account comprises integrating historical data collected by the first pulsatility sensor with data collected by the second pulsatility sensor.

47. The system of claim 44, wherein transitioning the account comprises integrating morphological data collected by the first pulsatility sensor with data collected by the second pulsatility sensor.

48. The system of claim wherein transitioning the account comprises integrating personalized settings associated with the first pulsatility sensor with personalized settings associated with the second pulsatility sensor.

49. The system of claim 44, wherein transitioning the account comprises integrating a user health profile associated with the first pulsatility sensor with a user health profile associated with the second pulsatility sensor.

50. The system of claim 33, wherein the first pulsatility sensor is configured to detect the health condition of the user and the second pulsatility sensor is configured to manage the health condition of the user.

51. The system of claim 33, wherein the health condition of the user is diagnosed based on the blood pressure value of the user.

52. The system of claim 33, wherein the first pulsatility sensor is one of a camera or a PPG sensor.

53. The system of claim 33, wherein the first pulsatility sensor comprises a video acquisition system.

54. The system of claim 33, wherein at least one of the first pulsatility sensor and the second pulsatility sensor is comprised in the user device.

55. A method for hypertension screening and monitoring, comprising:
collecting, from a user, at least one photoplethysmography (PPG) signal via a first pulsatility sensor;
processing the at least one PPG signal from the first pulsatility sensor to automatically generate a detection of a health condition of the user, the health condition comprising an identification of a hypertension risk; and
in response to detecting the health condition, automatically generating and causing a user device to display a notification,
wherein the notification comprises a recommendation for the user to use a second pulsatility sensor to obtain at least one pulsatility signal and process the at least one pulsatility signal to determine at least one blood pressure value.

56. The method of claim 55, wherein the first pulsatility sensor comprises a noninvasive optical sensor.

57. The method of claim 56, wherein the noninvasive optical sensor is one of a camera or a PPG sensor.

58. The method of claim 55, further comprising:
facilitating the user obtaining the second pulsatility sensor by collecting payment from the user for the second pulsatility sensor.

59. The method of claim 55, wherein the second pulsatility sensor is of a same type as the first pulsatility sensor.

60. The method of claim 55, wherein the second pulsatility sensor is of a different type than the first pulsatility sensor.

61. The method of claim 55, further comprising:
transitioning an account of the user from association with the first pulsatility sensor to association with the second pulsatility sensor.

62. The method of claim 61, wherein transitioning the account occurs without a need for the user to create a new account or download a separate application.

63. The method of claim 61, wherein transitioning the account comprises integrating historical data collected by the first pulsatility sensor with data collected by the second pulsatility sensor.

64. The method of claim 55, wherein the first pulsatility sensor is configured to detect the health condition of the user and the second pulsatility sensor is configured to manage the health condition of the user.

65. The method of claim 55, wherein the health condition of the user is diagnosed based on the blood pressure value of the user.
